(19) United States
(12) Patent Application Publication
(10) Pub. No.: US 2021/0145967 A1
(43) Pub. Date: May 20, 2021

(54) MAGNETIC LIQUID PARTICLES

(71) Applicant: ROYAL MELBOURNE INSTITUTE OF TECHNOLOGY, Melbourne (AU)

(72) Inventors: James Chapman, Melbourne (AU); Vi Khanh Truong, Melbourne (AU); Samuel Cheeseman, Melbourne (AU); Torben Jost Daeneke, Melbourne (AU); Aaron James Elbourne, Melbourne (AU)

(21) Appl. No.: 16/683,963

(22) Filed: Nov. 14, 2019

(51) Int. Cl. A61K 41/00; A61K 9/50; A61K 33/00; A01N 25/26; A01N 59/00

(52) U.S. Cl. CPC A61K 41/00; A61K 9/501; A01N 59/00; A01N 25/26; A61K 33/00

(57) ABSTRACT

The present disclosure generally relates to magnetic liquid particles, and methods for using the magnetic liquid particles. More specifically, the present disclosure relates to magnetic liquid particles having antimicrobial properties. The particles can comprise a liquid metal core comprising a liquid gallium or alloy thereof, and a plurality of magnetic iron particles; and an inorganic passivation layer encapsulating the liquid metal core. The particles can be used for disrupting a biofilm. The particles can also be used for the treatment of biofilm related diseases.

### FIELD

This disclosure relates to liquid metal particles which have antibacterial properties. This disclosure also relates to products and compositions comprising the liquid metal particles.

### BACKGROUND

A biofilm is community of microorganisms (including bacteria and fungi) within a self-produced three-dimensional matrix of extracellular polymeric substances (EPS). Biofilm formation is a critical step in pathogenesis. Once established, this three-dimensional matrix forms a protective environment for the microorganisms. Biofilm-related infections can be difficult to treat as the microorganisms present in the biofilm may become tolerant and/or resistant to antibiotics and the host's immune response. This means that antibiotic treatment alone is in often insufficient to eradicate biofilm infections. Biofilms contribute to patient morbidity and, due to their high frequency, their resistance to antibiotic treatment and the need to often remove infected medical devices to cure the infection cause significant health costs. It is thought that biofilms also contribute to the emergence and spread of antibiotic resistance. Accordingly, biofilms, and their associated infections pose a significant medical concern, often with life-threatening consequences.

Scientific and medical research has focused on the development of new therapeutic methods that are capable of treating biofilms. Initial research efforts have focussed on additive methods, which utilise antimicrobial or inhibitory agents, often incorporated within a surface to mitigate biofilm formation on the surface. However, these methods have not proved an attractive long-term option due to a number of disadvantages, such as patient tissue sensitivity, increasing antibiotic resistance, toxicity concerns and dosage complications. Furthermore, most of these approaches are passive, relying on the natural diffusion of therapeutic materials, or only mildly activated by a stimulus, such as light. More recently, the utilisation of nanostructured surfaces have emerged as an alternative method for minimising biofilm formation; however, these technologies are also passive in nature, and importantly, do not have the ability to disrupt an established biofilm.

Accordingly, there remains a clear need for novel agents having antimicrobial properties, and particularly agents that are suitable for use in treating biofilm related infections.

### SUMMARY

The present inventors have identified a liquid metal particle that has antimicrobial properties. In particular, the particles are able to disrupt biofilms comprising Gram-positive, Gram-negative bacteria and/or fungi, and can be considered as having broad spectrum activity. The present inventors have found that these particles are particularly suitable for treating established biofilms.

Accordingly, in a first aspect there is provided an antimicrobial particle comprising:

a liquid metal core comprising
a liquid gallium or alloy thereof, and
a plurality of magnetic iron particles; and

an inorganic passivation layer encapsulating the liquid metal core. In some embodiments, the particle is a microparticle or a nanoparticle. In some embodiments, the antimicrobial particle has a diameter of between 80 nm to 10 μm. In some embodiments, the particle is a sphere. In some embodiments, the particle becomes rod shaped, star shaped, spheroid shaped or a jagged shape in response to a magnetic field, such as a rotating magnetic field. In some embodiments, the particle fragments in response to a magnetic field, such as a rotating magnetic field.

In some embodiments, the gallium alloy comprises gallium and one or more metals selected from the group consisting of indium, tin, zinc, aluminium and copper. In some embodiments, the liquid metal core comprises an alloy of gallium and indium or an alloy of gallium, indium and tin or consists of gallium. In some embodiments, the liquid gallium or alloy thereof is eGaIn or Galinstan.

In some embodiments, the magnetic iron particles comprise Fe, Fe₃O₄, Fe₂O₃, γ-Fe₂O₃, or combinations thereof. In some embodiments, the magnetic iron particles comprise orthorhombic Fe I. In some embodiments, the magnetic iron particles are nanoparticles. In some embodiments, the magnetic iron particles have an average diameter of 35 nm to 1000 nm. In some embodiments, the magnetic iron particles have a concentration of between about 0.1% w/w and 10% w/w.

In some embodiments, the inorganic passivation layer comprises a metal oxide or a metal sub-oxide or a combination thereof. In some embodiments, the inorganic passivation layer comprises gallium oxide hydroxide (GaOOH) or gallium oxide (Ga₂O₃) or a combination thereof. In some embodiments, the inorganic passivation layer comprises at least 90% gallium oxide Ga₂O₃. In some embodiments, the inorganic passivation layer comprises at least 90% gallium oxide GaOOH. In some embodiments, the inorganic passivation layer has a thickness of between about 0.5 and 10 nm, for example between about 0.7 and 1.4 nm.

In another aspect, there is provided a composition comprising one or more antimicrobial particles according to any embodiments or examples thereof as described herein and a carrier fluid. In some embodiments, the composition is polydisperse. In some embodiments, the composition comprises at least one microparticle and at least one nanoparticle. In some embodiments, the carrier fluid is a pharmaceutically acceptable carrier fluid or a biocompatible carrier fluid. In some embodiments, the carrier fluid is water. In some embodiments, the composition further comprises at least one additional antimicrobial agent. In some embodiments, the concentration of the antimicrobial particles is between about 1 μg/mL and 1 mg/mL. In some embodiments, the concentration of the antimicrobial particles is about 100 μg/mL.

In yet another aspect, there is provided a method of disrupting a biofilm, the method comprising:

contacting the biofilm with the composition according to any embodiments or examples thereof as described herein; and

applying a magnetic field to the biofilm to magnetically activate the antimicrobial particles and thereby disrupt the biofilm. In another aspect, there is provided a method of treating a biofilm related disease in a subject, the method comprising administering to the subject the composition according to any embodiments or examples thereof as described herein; and applying a magnetic field to the subject. In one embodiment of any of the above aspects, the magnetic field is a rotating magnetic field. In some embodiments, the rotational speed of the magnet is between about 500 rpm and 2000 rpm. In some embodiments, the rotational speed of the magnet is about 1500 rpm. Further embodiments of any of the above aspects are described below.

In some embodiments, the magnetic field strength is between about 250 and 1500 milliGauss. In some embodiments, the magnetic field strength is about 775 milliGauss. In some embodiments, the magnetic field is located about 1 mm to 50 mm from the biofilm. In some embodiments, the magnetic field is located about 5 mm from the biofilm. In some embodiments, the magnetic field is applied for at least 5 minutes, at least 10 minutes, at least 20 minutes, at least 30 minutes, at least 60 minutes, at least 90 minutes or at least 120 minutes.

In some embodiments, the method further comprises contacting the biofilm simultaneously with an additional antimicrobial agent. In some embodiments, the method further comprises contacting the disrupted biofilm with an additional antimicrobial agent. In some embodiments, the biofilm is located on or in a medical device or portion thereof.

In some embodiments, the biofilm is formed from bacteria and/or fungi. In some embodiments, the biofilm is formed from bacteria of the genus Actinobacillus, Acinetobacter, Aeromonas, Bordetella, Brevibacillus, Brucella, Bacteroides, Burkholderia, Borelia, Bacillus, Campylobacter, Capnocytophaga, Cardiobacterium, Citrobacter, Clostridium, Chlamydia, Eikenella, Enterobacter, Escherichia, Entembacter, Francisella, Fusobacterium, Flavobacterium, Haemophilus, Helicobacter, Kingella, Klebsiella, Legionella, Listeria, Leptospirae, Moraxella, Morganella, Mycoplasma, Mycobacterium, Neisseria, Pasteurella, Proteus, Prevotella, Plesiomonas, Pseudomonas, Providencia, Rickettsia, Stenotrophomonas, Staphylococcus, Streptococcus, Streptomyces, Salmonella, Serratia, Shigella, Spirillum, Treponema, Veillonella, Vibrio, Yersinia, or Xanthomonas. In some embodiments, the bacteria is Escherichia coli, Pseudomonas aeruginosa, Staphylococcus aureus, Bacillus cereus, or combinations thereof. In some embodiments, the biofilm is formed from fungi of the genus Candida, Aspergillus, Cryptococcus, Trichosporon, Coccidioides, or Pneumocystis. In some embodiments, the comprises Candida or Crytococcus or a combination thereof. In some embodiments, the fungi comprises Candida albicans. In some embodiments, the fungi comprises Crytococcus neoformans.

In yet another aspect, there is provided a process for forming a composition comprising antimicrobial particles, the process comprising:

(i) combining a liquid metal comprising gallium or an alloy thereof with magnetic iron particles to form a liquid metal ferrofluid, and

(ii) sonicating the liquid metal ferrofluid in an aqueous carrier fluid to form the antibacterial particles, wherein the antimicrobial particle comprises

a liquid metal core comprising
a liquid gallium or alloy thereof, and
a plurality of magnetic iron particles, and

an inorganic passivation layer encapsulating the liquid metal core. In some embodiments, step (i) comprises grinding the liquid metal comprising gallium or an alloy thereof with magnetic iron particles under an inert atmosphere. In some embodiments, the grinding is carried out using a mortar and pestle.

In some embodiments, the liquid metal ferrofluid comprises about 0.1% w/w to 10% w/w magnetic iron particles. In some embodiments, the aqueous carrier fluid is water.

In some embodiments, the sonicating is carried out for between 5 minutes and 30 minutes. In some embodiments, the sonicating is carried out at a temperature less than 40° C., or less than 30° C., or less than 25° C. In some embodiments, the sonicating is carried out at a frequency of between 60 Hz and 60 kHz. In some embodiments, the sonicating is carried out with sonication intensity of about 10%. In some embodiments, the sonicating is carried out with a probe diameter of between 3.7 mm to 41 mm. In some embodiments, the sonicating is carried out at a power of between 60 watts and 240 watts.

### BRIEF DESCRIPTION OF DRAWINGS

FIG. 1. Schematic representation of the preparation of antimicrobial particles in accordance with an embodiment of the present application. In this embodiment, antimicrobial particles are prepared by sonicating a liquid metal ferrofluid in an aqueous carrier fluid.

FIG. 2. Characterization of exemplified antimicrobial particles (GLM-Fe particles). A) DLS data obtained for GLM-Fe particles in solution. B) An 8 μm×8 μm AFM image revealed a cluster of GLM-Fe particles adsorbed onto a mica surface (black background). C) High resolution transmission electron microscopy (HRTEM) image of an isolated GLM-Fe particle. D) HRTEM image of a GLM-Fe particle revealed the atomic lattice. E) Higher magnification image of the atomic lattice. The 2D-fast Fourier transform (FFT) data is shown as an inset. F) Scanning electron microscope (SEM) image of GLM-Fe particles obtained following sonication deposited on a bare silicon surface. G) High-magnification SEM image of the central particles of FIG. 2G. Arrows indicate nano-fragments deposited on the silicon surface and attached to the periphery of the larger particles. H) HRTEM image of an isolated GLM-Fe particle where the internalized Fe could be observed central to the particle. Arrows indicate nano-fragments deposited on the silicon surface and attached to the periphery of the larger particles. I) Magnified section of FIG. 21. J) Corresponding 2D-FFT to FIG. 2J revealed the orthorhombic 001 and 002 planes of the atomic lattice of the encapsulated iron.

FIG. 3. High resolution XPS spectra of an exemplified antimicrobial particle. A) pre-magnetised and B) post-magenetised GLM-Fe particles drop cast onto a clean silicon substrate. Peak positions and binding energy ranges were auto selected by the Avantage software. Peaks were assigned in accordance with the Avantage database, Ga0 peaks are located at 18.7 eV (Ga 3d), 159.5 eV (Ga 3s) and 1117 eV (Ga 2p) eV. In0 and Sn0 peaks are observed at 444 eV (In 3d) and 484.8 eV (Sn 3d), respectively. For the Ga 3d data, the experimental data, the general fit, the Ga3d_{5/2} (Element), the Ga3d_{3/2} (Element) and the Ga3d₃ (Native Oxide) are labelled. For the Ga 2p data, the experimental data, the general fit and the Ga2p1/2 (Native Oxide) are labelled.

FIG. 4. EDX spectra of an exemplified antimicrobial particle. A) pre-magnetised and B) post-magnetised GLM-Fe particles drop cast onto a clean silicon substrate. The respective SEM images are shown alongside the EDX maps of Gallium (Ga), Indium (In), Oxygen (O), Tin (Sn), and Iron (Fe).

FIG. 5. High-resolution microscopic investigation of exemplified antimicrobial particles post-magnetisation. The particles were observed to adopt three main morphological categories following magnetisation—A) Spheroids, B) Rods, and C) stars. Representative SEM (left) and TEM (right) images display the variant morphologies of post-magnetised particles. The HRTEM images highlight nanoparticles with thin, extruded asperities (highlighted by the arrows). The inset to FIG. 5C, right-middle shows a star-shaped nanoparticle edge, revealing a nanosheet of material. The inset shows the 2D-FFT of the atomic lattice. It is thought that these asperities are only several atomic layers thick, and are therefore nano-sharp D) Histogram displaying the aspect ratio of the spherioid, rod, and star shaped particles as calculated from both TEM and SEM images. E) Representative high resolution AFM images of the nanoparticles. A high profile taken from the region under the white line indicates that the particles have a rough, sharp exterior surface. F) Histogram of the heights of various asperities measured form the side of particles using AFM profiling of 75 particles. G) DLS data obtained for the post-magnetised particles in solution. I) Pictorial representation of the magnetically induced shape transformation of the exemplified antimicrobial particles.

FIG. 6. Characterisation of an exemplified antimicrobial particle post-magnetization. SEM micrographs displaying the variant morphologies of post-magnetised GLM-Fe particles. The particles could largely be placed into three morphological categories, including rods, spheroids, and stars. The white scale bar is 200 nm in each image.

FIG. 7. Treatment of bacterial biofilms embedded with exemplified antimicrobial particles and a magnetic field. (A and B): Low magnification top-down SEM micrographs of the A) P. aeruginosa and B) S. aureus biofilms following 24 hours of growth with the GLM-Fe solution. GLM-Fe particles imbedded within the biofilm are highlighted by the dark grey arrows. The black arrows highlight areas of strong EPS growth within the biofilm of P. aeruginosa in image (A). (C and D): Biofilms of both bacterial species (indicated above the image) following 90 minutes of exposure to the rotating magnetic field. A distinct decrease in the number of surface attached cells could be observed in both images. (E, I, G and K): High-magnification SEM and TEM images of control cells for P. aeruginosa (E and I) and S. aureus (G and K) revealing healthy, intact cells. (F, G, H and L): High-magnification SEM and TEM images of P. aeruginosa (F and J) and S. aureus (H and L) following 90 minutes of magnetic field exposure. Physical damage to the bacterial membrane can be observed in all images following magnetic activation.

FIG. 8. Example magnetic treatment system. A) Schematic representation of an exemplified magnetic treatment system. B) Ferrite rare earth magnet used, for example, for general antimicrobial particle activation C) Neodymium magnet used, for example, for targeted treatment.

FIG. 9. Treatment of bacterial biofilms embedded with exemplified antimicrobial particles and a magnetic field. Additional SEM micrographs of P. aeruginosa (left panel) and S. aureus cells (right panel) following 90 minutes of treatment with the rotating magnetic fields in the presence of GLM-Fe particles. The white scale bars are 500 nm.

FIG. 10. Antibacterial response of exemplified antimicrobial particles to bacterial biofilms as a function of magnetic exposure. CLSM images of (A-D) Pseudomonas aeruginosa and (E-H) Staphylococcus aureus biofilms treated with GLM-Fe particle solution following 30 min increments of magnetic field exposure. The magnetic exposure time is indicated to the left of the respective images. The CLSM images are 220 μm×220 μm, with the relative thickness indicated next to images in E) and I).

FIG. 11. Quantification of bacterial biofilms after the treatment of exemplified antimicrobial particles as a function of magnetic exposure. (A) Average number of inactivated cells expressed as a percentage and (B) Biofilm biomass following the incremental magnetic field exposure corresponding to FIG. 10 expressed as a percentage of initial mass. (C) Raw biofilm mass (μm³/μm²) as a function of magnetic activation.

FIG. 12. Treatment of bacterial biofilms with exemplified antimicrobial particles and local magnetic field of varying strength. P. aeruginosa and S. aureus biofilms were treated with either a small or larger magnet, then stained with crystal violet. The zoomed inset shows a CLSM image of the periphery of the treated area. Viable biofilm (light grey are within the inset) was observed in the untreated area, while only inactivated cells (dark grey/black area within the inset) and a significantly diminished biofilm mass were seen inside the treated area.

FIG. 13. Antimicrobial performance of exemplified antimicrobial particles. CLSM images of P. aeruginosa and S. aureus biofilms after treatment with Galistan particles (GLM), exemplified particles (GLM-Fe particles), or pre-magnetised GLM-Fe particles followed by incubation in the absence or presence of 90 min magnetic exposure. The particles and subsequent exposure conditions are indicated to the left of the respective images. The CLSM images are 220 μm×220 μm.

FIG. 14. Antimicrobial performance of exemplified antimicrobial particles. TEM images of bacteria co-cultured with exemplified antimicrobial particles (GLM-Fe particles) in the absence of magnetic field. There was no sign of cellular damage or particles entering the cells.

FIG. 15. Cytotoxicity assessment of exemplified antimicrobial particles. Assessment of cytotoxicity of GLM particles, exemplified particles (GLM-Fe particles), and pre-magnetised GLM-Fe particles on HEK cell lines. A) The data shows the viability of HEK cells in the presence of particles (100 μg/mL) after 2 days of incubation against control samples (with no introduction of particles) with and without magnetisation for 90 minutes. B) Assessment of the innate cytotoxicity of the GLM and GLM-Fe particles without magnetisation as a function of concentration. The negative control is cells grown without the presence of any particles, and the positive control SDS and Triton X-100 (0.1 wt %/vol) were included to show the efficacy of the AlamarBlue assay. These data were compared with the untreated HEK cells and expressed in terms of the cell viability (%). Each experiment was repeated three times.

FIG. 16. Cytotoxicity of exemplified antimicrobial particles. Optical phase contrast images showing no inhibition of HEK cell growth after treatment with GLM, GLM-Fe or magnetically activated GLM-Fe. Despite the increase in the concentration of the respective particles, HEK cells were shown to be able to proliferate and differentiate. Under exposure to magnetic field, HEK cells continued to grow healthily after 2 days of incubation (the duration of observation). The white scale bar is 100 μm.

FIG. 17. Inactivation of microbial cells by exemplified antimicrobial particles. Schematic representation of the physical action of the GLM-Fe particles causing the inactivation of microbial cells and reduction in the biofilm volume. 1. Planktonic cell attachment of viable microbial cells (grey). 2. Active biofilm being treated with GLM-Fe particles. 3. Magnetic activation of the GLM-Fe particles simultaneously disrupts the biofilm matrix, while physically inducing microbial cell lysis, producing deactivated cells (black). 4. The treated area had a lower biofilm mass, with the majority of cells being deactivated.

### DETAILED DESCRIPTION

### General Definitions

Unless specifically defined otherwise, all technical and scientific terms used herein shall be taken to have the same meaning as commonly understood by one of ordinary skill in the art (e.g., chemistry, biochemistry, medicinal chemistry, microbiology and the like). With regards to the definitions provided herein, unless stated otherwise, or implicit from context, the defined terms and phrases include the provided meanings. Unless explicitly stated otherwise, or apparent from context, the terms and phrases below do not exclude the meaning that the term or phrase has acquired by a person skilled in the relevant art. The definitions are provided to aid in describing particular embodiments, and are not intended to limit the claimed invention, because the scope of the invention is limited only by the claims.

Unless otherwise indicated, the cell culture and microbiology techniques utilized in the present disclosure are standard procedures, known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T.A. Brown (editor), Essential Molecular Biology: A Practical Approach, Volumes 1 and 2, IRL Press (1991), D.M. Glover and B.D. Hames (editors), DNA Cloning: A Practical Approach, Volumes 1-4, IRL Press (1995 and 1996), and F.M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J.E. Coligan et al. (editors) Current Protocols in Immunology, John Wiley & Sons (including all updates until present).

In the following description, reference is made to the accompanying drawings which form a part hereof, and which is shown, by way of illustration, several embodiments. It is understood that other embodiments may be utilized and structural changes may be made without departing from the scope of the present disclosure.

All publications discussed and/or referenced herein are incorporated herein in their entirety.

Any discussion of documents, acts, materials, devices, articles or the like which has been included in the present specification is solely for the purpose of providing a context for the present disclosure. It is not to be taken as an admission that any or all of these matters form part of the prior art base or were common general knowledge in the field relevant to the present disclosure as it existed before the priority date of each claim of this application.

Throughout this disclosure, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or groups of compositions of matter. Furthermore, unless otherwise required by context, singular terms shall include pluralities and plural terms shall include the singular. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the disclosure herein is susceptible to variations and modifications other than those specifically described. It is to be understood that the disclosure includes all such variations and modifications. The disclosure also includes all of the examples, steps, features, methods, compositions, coatings, processes, and coated substrates, referred to or indicated in this specification, individually or collectively, and any and all combinations or any two or more of said steps or features.

As used herein, the term “and/or”, e.g., “X and/or Y” shall be understood to mean either “X and Y” or “X or Y” and shall be taken to provide explicit support for both meanings or for either meaning, e.g. A and/or B includes the options i) A, ii) B or iii) A and B.

Unless otherwise indicated, the terms “first,” “second,” etc. are used herein merely as labels, and are not intended to impose ordinal, positional, or hierarchical requirements on the items to which these terms refer. Moreover, reference to a “second” item does not require or preclude the existence of lower-numbered item (e.g., a “first” item) and/or a higher-numbered item (e.g., a “third” item).

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example and without limitation, two of item A, one of item B, and ten of item C; four of item B and seven of item C; or some other suitable combination.

As used herein, the term “about”, unless stated to the contrary, typically refers to +/−10%, for example +/−5%, of the designated value.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout the present specification, various aspects and components of the invention can be presented in a range format. The range format is included for convenience and should not be interpreted as an inflexible limitation on the scope of the invention. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 5, 5.5 and 6, unless where integers are required or implicit from context. This applies regardless of the breadth of the disclosed range. Where specific values are required, these will be indicated in the specification.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The reference to “substantially free” generally refers to the absence of that compound or component in the composition other than any trace amounts or impurities that may be present, for example this may be an amount by weight % in the total composition of less than about 1%, 0.1%, 0.01%, 0.001%, or 0.0001%. The compositions as described herein may also include, for example, impurities in an amount by weight % in the total composition of less than about 5%, 4%, 3%, 2%, 1%, 0.5%, 0.1%, 0.01%, 0.001%, or 0.0001%. An example is the amount of water that may be present in an organic solvent.

As used herein, bacteria or fungi are referred to in both the singular and plural. In particular they are referred to in the singular when defining the type of microorganism to be targeted (i.e. the type, e.g. species) and in the plural when referring to the treatment to which they may be subjected (i.e. treatment of multiple microorganisms).

As used herein, the term “biofilm”, “microbial biofilm”, or like term, refers a community of microorganisms within a three-dimensional matrix of extracellular polymeric substances (EPS). Microorgamisms that are capable of forming a biofilm include, but are not limited to, bacteria, fungi, yeast, protozoa, and the like. For example, the biofilm may be formed from bacteria and/or fungi according to any one or more of the embodiments or examples as described herein.

As used herein, the term “treat”, “treated”, “treatment”, “treating” or like terms when used with respect to a disease or disorder, such as a biofilm related disease refers to a therapeutic or prophylactic treatment that increases the resistance of a subject to development of the disease (e.g., to infection with a pathogen, such as a bacteria or fungus), that decreases the likelihood that the subject will develop the disease (e.g., become infected with the pathogen), that increases the ability of a subject that has developed disease (e.g., a pathogenic (e.g., fungal) infection) to fight the disease (e.g., reduce or eliminate at least one symptom typically associated with the infection) or prevent the disease from becoming worse, or that decreases, reduces, or inhibits at least one function of the pathogen (e.g., a fungus, such as Candida albicans), such as form a biofilm, and/or to grow by at least 10% (e.g., at least 20%, 30%, 40%, 50%, 60%, 70%, 80%, 90%, 95%, or 100%). In some embodiments, “treat,” “treated,” “treatment” or “treating” refers to a therapeutic or prophylactic treatment that disrupts a biofilm or part thereof and/or increases the ability of a subject that has developed disease (e.g., a pathogenic (e.g., fungal) infection) to fight the disease (e.g., reduce or eliminate at least one symptom typically associated with the infection).

### An Antimicrobial Particle

In one aspect, there is provided an antimicrobial particle comprising:

a liquid metal core comprising
a liquid gallium or alloy thereof, and
a plurality of magnetic iron particles; and

an inorganic passivation layer encapsulating the liquid metal core.

The antimicrobial particles described herein comprise a liquid metal core. As used herein, the term “liquid metal” refers to a metal or metal alloy having a melting point of less than 40° C., less than 35° C., less than 30° C., less than 25° C. or less than 20° C. Generally, the melting point of the metal or metal alloy is approximately room temperature or below. This means that the metal or metal alloy remains in the liquid state at room temperature. Typically, francium (Fr), caesium (Cs), rubidium (Rb), mercury (Hg) and gallium (Ga) can be defined as liquid metals. However, the properties of gallium mean that it is particularly suited to use in bio-applications. Furthermore, gallium-based liquid metals are reported to have good biodegradability under physiological conditions and low toxicity in mouse models.

In the antibacterial particles described herein, the liquid metal core comprises a liquid gallium or an alloy thereof. In some embodiments, the liquid gallium or alloy thereof comprises gallium. In some embodiments, the liquid gallium or alloy thereof consists of gallium (i.e. pure gallium). The reference to “pure” material generally refers a material that comprises other compounds or components in trace amounts or as an impurity, For example, a “pure” material may comprise an amount by weight % of the total composition of less than about 1%, 0.1%, 0.01%, 0.001%, or 0.0001% of other compounds or components. As used herein, pure gallium comprises at least about 99% w/w gallium, at least about 99.9% w/w gallium, or at least about 99.99% w/w gallium. The remainder typically comprises copper, iron, germanium, indium, lead, tin and/or zinc. Pure gallium has a melting point of about 29.7° C.

In some embodiments, the liquid gallium or alloy thereof comprises a gallium alloy. In some embodiments, the liquid gallium or alloy thereof consists of a gallium alloy. Suitable alloys are non-toxic, and/or have minimum toxicity to humans and other subjects and/or are suitable for use in bio-applications. In some examples, the gallium alloy comprises gallium and one or more metals selected from the group consisting of indium, tin, zinc, aluminium and copper. In some examples, the gallium alloy comprises gallium and one or more metals selected from indium, tin and zinc. In some examples, the gallium alloy comprises gallium and one or more metals selected from indium and tin. In some examples, the liquid metal core comprises an alloy of gallium and indium, an alloy of gallium and tin or an alloy of gallium, indium and tin. In one example, the liquid metal core comprises an alloy of gallium and indium. In one example, the liquid metal core comprises an alloy of gallium and tin. In one example, the liquid metal core comprises an alloy of gallium, indium and tin.

As the person skilled in the art would understand, a eutectic composition of an alloy is a composition having the ratio of the elements which allow the alloy to melt congruently at a single melting point that is lower than the melting point of the separate components. Alloys having a composition that deviates from the eutectic composition may form monophasic liquid metals at higher temperatures (c.f. the melting point of the eutectic composition). In some embodiments, the gallium alloy useful herein is a eutectic gallium alloy. However, in some embodiments the gallium alloy may have a composition that deviates from the eutectic composition provided the alloy is a liquid metal, for example is a liquid under ambient conditions. In some embodiments, the liquid metal core comprises or consists of eGaIn, for example a gallium alloy having about 85.8 wt % Ga and about 14.2 wt % In. In some embodiments, the liquid metal core comprises or consists of eGaSn, for example a gallium alloy having about 91.7% wt % Ga and about 8.3 wt % Sn. Other examples of binary gallium alloys are provided in Daeneke, T., et al., Chem. Soc. Rev., 2018, 47, 4073. In some embodiments, the liquid metal core comprises or consists of eGaInSn, for example a gallium alloy having about 78.3% wt % Ga, 14.98 wt % In and about 6.8 wt % Sn. eGaInSn is also referred to as Galinstan.

The liquid metal core also comprises magnetic iron particles. Generally, the iron particles are not dissolved in the liquid metal and form a second phase. Therefore, in some embodiments, the liquid metal core can also be referred to as a biphasic liquid metal, i.e. comprising a liquid metal and solid particles. In some embodiments, the liquid gallium or alloy thereof and magnetic iron particles form a liquid metal ferrofluid.

Any suitable magnetic iron particle may be used. In some embodiments, the magnetic iron particles comprise iron or iron oxide or a combination thereof. In some embodiments, the magnetic iron particles are selected from the group consisting of Fe, Fe₃O₄, Fe₂O₃, γ-Fe₂O₃ and combinations thereof. In some embodiments, the magnetic iron particles are Fe. In some embodiments, the magnetic iron particles comprise orthorhombic Fe, e.g. orthorhombic Fe I.

The average diameter of the magnetic iron particles is such that the magnetic iron particles remain suspended in the liquid metal. In some embodiments, the magnetic iron particles have an average diameter of less than about 1000 nm, less than about 900 nm, less than about 800 nm, less than about 700 nm, less than about 600 nm, less than about 500 nm, less than about 400 nm, less than about 300 nm, less than about 200 nm, less than about 100 nm, less than about 75 nm or less than about 50 nm. In some embodiments, the magnetic iron particles have an average diameter of greater than about 10 nm, about 20 nm, about 30 nm, about 40 nm, about 50 nm, about 60 nm, about 70 nm, about 80 nm, about 90 nm, about 100 nm, about 200 nm, about 300 nm, about 400 nm, or about 500 nm. The diameter may also be provided in a range between any two of these upper and/or lower values. In some embodiments, the magnetic iron particles have an average diameter of between about 35 nm to 1000 nm, 35 nm to 900 nm, 35 nm to 800 nm, 35 nm to 700 nm, 35 nm to 600 nm, 35 nm to 500 nm, 35 nm to 400 nm, 35 nm to 300 nm, 35 nm to 200 nm or 35 nm to 100 nm. In some examples, the magnetic iron particle is a nanoparticle.

The concentration of the magnetic iron particle is such that the magnetic iron particle remains suspended in the liquid metal. In some embodiments, the magnetic iron particles have a concentration of between about 0.1% w/w and 10% w/w, for example 0.1, 1, 2, 3, 4, 5, 6, 7, 8, 9 or 10 wt %. In some embodiments, the magnetic iron particles have a concentration of between about 0.1% w/w and 10% w/w, 0.1% w/w and 9% w/w, 0.1% w/w and 8% w/w, 0.1% w/w and 7% w/w, 0.1% w/w and 6% w/w, 0.1% w/w and 5% w/w, 0.1% w/w and 4% w/w, 0.1% w/w and 3% w/w, 0.1% w/w and 2% w/w, 0.1% w/w and 1% w/w, 1% w/w and 10% w/w, 2% w/w and 10% w/w, 3% w/w and 10% w/w, 4% w/w and 10% w/w, 5% w/w and 10% w/w, 6% w/w and 10% w/w, 7% w/w and 10% w/w, 8% w/w and 10% w/w or 9% w/w and 10% w/w. In some embodiments, the magnetic iron particles have a concentration of about 5% w/w.

The antibacterial particles described herein also comprise an inorganic passivation layer encapsulating the liquid metal core. In this context, the term “inorganic” refers to non-carbon based materials. As used herein, the term “encapsulating” refers to enclosing a substance (i.e. the liquid metal core) with an layer of material. As used herein, a “passivation layer” is a layer of material formed from reaction of the liquid metal with an oxidiser, for example a layer that forms as the result of a self-terminating Cabrera-Mott oxidation mechanism. In some embodiments, the “inorganic passivation layer” is formed by contacting the liquid metal with a suitable oxidiser under conditions suitable for formation of the particles (e.g. sonication). In some embodiments, the liquid metal spontaneously self-encapsulates within a Cabrerra-Mott oxide layer during exposure to an oxidiser. Non limiting examples of oxidisers include water and oxygen.

In some embodiments, the inorganic passivation layer comprises a metal oxide or a metal sub-oxide or a combination thereof. In some embodiments, the inorganic passivation layer comprises gallium (III). In some embodiments, the inorganic passivation layer comprises gallium oxide hydroxide (GaOOH) or gallium oxide Ga₂O₃ or a combination thereof. In some embodiments, the inorganic passivation layer comprises gallium oxide hydroxide (GaOOH). In some embodiments, the inorganic passivation layer comprises at least 10 wt %, at least 20 wt %, at least 30 wt %, at least 40 wt %, at least 50 wt %, at least 60 wt %, at least 70 wt %, at least 80 wt %, at least 90 wt %, at least 95 wt %, at least 98 wt %, at least 99 wt % gallium oxide hydroxide. In some embodiments, the inorganic passivation layer consists of gallium oxide hydroxide (GaOOH). In some embodiments, the inorganic passivation layer comprises gallium oxide (Ga₂O₃). In some embodiments, the inorganic passivation layer comprises at least 10 wt %, at least 20 wt %, at least 30 wt %, at least 40 wt %, at least 50 wt %, at least 60 wt %, at least 70 wt %, at least 80 wt %, at least 90 wt %, at least 95 wt %, at least 98 wt %, at least 99 wt % gallium oxide. In some embodiments, the inorganic passivation layer consists of gallium oxide (Ga₂O₃). For gallium and its eutectic alloys, the inorganic passivation layer is thought to separate and help sustain individual liquid particles, so that they do not significantly aggregate.

Generally, the thickness of the inorganic passivation layer is suitable to maintain the integrity of the antimicrobial particle in the absence of a magnetic field. The thickness of the inorganic passivation layer can be determined by techniques known to the person skilled in the art. In some embodiments, the inorganic passivation layer has a thickness of between 0.5 and 10 nm. In some embodiments, the thickness of the inorganic passivation layer is least about (in nm) 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0. In some embodiments, the thickness of the inorganic passivation layer is less than about (in nm) 10, 9, 8, 7, 6, 5, 4, 3, 2.5, 2, 1.9, 1.8, 1.7, 1.6, 1.5, 1.4, 1.3, 1.2, 1.1, 1.0, 0.9, 0.8, or 0.7. In some embodiments, the inorganic passivation layer has a thickness of between about 0.6 and 2 nm. In some embodiments, the inorganic passivation layer has a thickness of between about 0.7 and 1.4 nm. In some embodiments, the inorganic passivation layer is several atoms thick. In some embodiments, the thickness of the inorganic passivation layer can be modulated by using an electrochemical method.

In some embodiments, the antimicrobial particle further comprises an organic layer. In some embodiments, the antimicrobial particle does not comprise an organic layer. For example, the inorganic passivation layer may consist of one or more inorganic layers according to any embodiments or examples thereof as described herein. In some embodiments, the antimicrobial particle does not comprise an outer organic layer. In some embodiments, the antimicrobial particle consists of:

a liquid metal core comprising
a liquid gallium or alloy thereof, and
a plurality of magnetic iron particles; and

an inorganic passivation layer encapsulating the liquid metal core.

As used herein, the term “organic layer” refers to a layer comprising an organic (i.e. carbon based) material. Organic materials include, but are not limited to proteins, nucleic acids, carboxylic acids and the like. In some embodiments, the organic material is a carboxylic acid or is derived from a carboxylic acid. Non-limiting examples of carboxylic acids include saturated aliphatic carboxylic acids having one to 20 carbon atoms such as formic acid, acetic acid, propanoic acid, butyric acid, hexanoic acid, heptanoic acid, octanoic acid, decanoic acid, and higher aliphatic acids such as hexadecanoic acid and octadecanoic acid. In some embodiments, the organic material is acetate.

In some embodiments, the antimicrobial particle (prior to exposure to a magnetic field) has a diameter of between 80 nm and 10 μm, for example, about 80 nm, 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm or 10 μm, although smaller and larger particles are within the scope of this disclosure. In some embodiments, the antimicrobial particle has an average diameter of between 80 nm and 5 μm, or between 200 nm and 2 μm. The average diameter of the antimicrobial particle can be determined by techniques known to the person skilled in the art, for example dynamic light scattering, scanning electron microscopy, atomic force microscopy or transmission electron microscopy. The surface area to volume ratio may be increased using techniques known to the person skilled in the art, such as ultrasonication

In some embodiments, the antimicrobial particle is a microparticle or a nanoparticle. As used herein, the term “microparticle” means particles having a diameter between about 0.1 μm and 100 μm, for example greater than about 100 nm. As used herein, the term “nanoparticle” means particles having a diameter between about 1 nm and 100 nm, for example greater than about 1 nm. As would be understood by a person skilled in the art, particles are three dimensional. Accordingly, where the nanoparticles or micro particles do not have a uniform shape (for example, a rod, star, oval and the like) at least two of the three dimensions should be between 1 nm and 100 nm. For example, a nanotube with a diameter of 10 nm and a length of greater than 100 nm is considered a nanoparticle.

In some embodiments, the antimicrobial particles may be self-repairing. For example, if the inorganic passivation layer is punctured, scratched, or otherwise breached, then it may quickly reform and thereby “re-seal” the particle. Without wishing to be bound by theory it is thought that this self-repairing characteristic may be due to the fact that the particle is in the presence of oxygen, which, as discussed above, may readily react with the liquid metal core to form a metal oxide.

In some embodiments, the antimicrobial particle is a sphere or has a sphere like shape prior to exposure of the particle to a magnetic field, although antimicrobial particle may also form other shapes. In response to a magnetic field (e.g. rotating magnetic field) the antimicrobial particle is capable of changing shape and/or size. In some embodiments, the antimicrobial particle becomes rod shaped, star shaped, spheroid shaped or a jagged sphere after exposure to a magnetic field (e.g. rotating magnetic field).

In some embodiments, the antimicrobial particle forms asperities in response to a magnetic field. In some embodiments, the asperities comprise nanosheets. As used herein, a “nanosheet” is a two-dimensional nanostructure with thickness in a scale ranging from 1 to 100 nm. In some embodiments, the nanosheet comprises a single layer of GaOOH and/or Ga₂O₃. In some embodiments, the nanosheet comprises at least two layers of GaOOH and/or Ga₂O₃, for example, two layers, three layers, four layers or five layers. Without wishing to be bound by theory, it is thought that the asperities can behave as a nano-knife and pierce the cellular membrane potentially causing the microorganism to rupture/lyse.

In some embodiments, the antimicrobial particle is capable of fragmenting in response to a magnetic field (e.g. rotating magnetic field). Generally, after exposure to the magnetic field the average diameter of the antimicrobial particle decreases. In some embodiments, the antimicrobial particle after exposure to a magnetic field has a diameter of between 10 nm and 10 μm, for example, about 10 nm, 20 nm, 30 nm, 40 nm, 50 nm, 60 nm, 70 nm, 80 nm, 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm or 10 μm. In some embodiments, the antimicrobial particle has an average diameter of between 10 nm and 5 μm, or between 10 nm and 2 μm.

Generally, the antimicrobial particles described herein have antimicrobial activity. As used herein, the term “antimicrobial activity” is defined broadly and refers to the property or capability of a particle to disrupt biofilms and/or inactivate microorganisms. Generally, this “inactivation” renders the microorganism non-viable (e.g. incapable of growth and/or reproduction) and occurs by disruption of the microorganism's membrane. Non-limiting examples of microorganisms include bacteria and fungi. Antimicrobial particles includes particles having antibacterial and/or antifungal activity. In some embodiments, the antimicrobial particle has antibacterial activity. In some embodiments, the antimicrobial particle has antifungal activity. In some embodiments, the antimicrobial particle has antibacterial activity and antifungal activity.

In some embodiments, the antimicrobial particles have broad spectrum antimicrobial activity. In some embodiments, the antimicrobial particles have broad spectrum antibacterial activity. In some embodiments, the antimicrobial particles have broad spectrum antifungal activity. As used herein, the term “broad spectrum” refers to the property or capability of the particle to inactivate numerous different, or substantially all, types of the microorganism. For example, “broad spectrum” antibacterial activity means the particles inactivate numerous different, or substantially all, types of bacteria. An antibacterial agent that inactivates only one or a subset of bacterial species does not have broad spectrum antimicrobial activity. In some examples as described herein, broad spectrum refers an antimicrobial that acts on Gram-positive and Gram-negative bacteria, or an antimicrobial that acts against a wide range of disease-causing bacteria.

### Composition

Generally, one or more antibacterial particle(s) as described herein are presented as a composition. Accordingly, in another aspect there is provided a composition comprising one or more antimicrobial particles according to any embodiments or examples thereof as described herein and a carrier fluid. In some embodiments, the composition is a pharmaceutical composition.
In some embodiments, the average diameter of the antimicrobial particles (prior to exposure to a magnetic field) in the composition is between 80 nm and 10 μm, for example, about 80 nm, 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm or 10 μm, although smaller and larger particles are within the scope of this disclosure. In some embodiments, the antimicrobial particles have an average diameter of between 80 nm and 5 μm, or between 200 nm and 2 μm.

The compositions described herein comprise one or more antibacterial particles. In some embodiments, the composition is polydisperse. In some embodiments, the composition comprises at least one microparticle and at least one nanoparticle. The present inventors have surprisingly found that further advantages can be provided by a composition comprising at least one microparticle and at least one nanoparticle, for example improved capability in disrupting a biofilm and/or lysing cells.

Any suitable carrier fluid carrier can be used. As the person skilled in the art would understand the carrier fluid should be compatible with the end use. In some embodiments, the carrier fluid is a pharmaceutically acceptable carrier fluid or a biocompatible carrier fluid. In some embodiments, the carrier fluid is the identical to the aqueous carrier fluid used to form the antimicrobial particles (for example ultrapure or MilliQ water). In some embodiments, the carrier fluid is different to the aqueous carrier fluid used to form the antimicrobial particles. If the carrier fluid is different to the aqueous carrier fluid used to form the antimicrobial particles, the carrier fluid can be exchanged for the aqueous carrier fluid using techniques known to the person skilled in the art, for example, buffer exchange, dialysis, desalting and the like.

In some embodiments, the carrier fluid (also referred to as a “carrier”) is pharmaceutically acceptable in the sense of being compatible with the other ingredients of the composition and not unduly deleterious to the recipient thereof. Generally, suitable pharmaceutically acceptable carriers are known in the art and are selected based on the end use application. The pharmaceutically acceptable carrier may act as a diluent, dispersant or carrier for the antibacterial particles and other optional components of the composition. The pharmaceutically acceptable carrier may also contain materials commonly used in pharmaceutically products and can be in a wide variety of forms. For example, the carrier may be water, liquid or solid emollients, silicone oils, emulsifiers, surfactants, solvents, humectants, thickeners, powders, propellants and the like. In some embodiments, the carrier fluid is a solvent, such as water or a pharmaceutically acceptable organic solvent. In some embodiments, the carrier fluid is an aqueous fluid (e.g. water). In some embodiments, the water is ultrapure water, such as MilliQ water.

In some embodiments, the composition further comprises one or more excipients and/or other additives, for example one or more pharmaceutically acceptable excipients and/or other additives. Generally, suitable excipients and/or other additives are known in the art and are selected based on the end use application. The compositions may further include, for example, diluents, buffers, citrate, trehalose, binders, disintegrants, thickeners, lubricants, preservatives (including antioxidants), inorganic salts (e.g., sodium chloride), antimicrobial agents (e.g., benzalkonium chloride), sweeteners, antistatic agents, sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). The compositions of the present disclosure may also include polymeric excipients/additives or carriers, e.g., polyvinylpyrrolidones, derivatised celluloses such as hydroxymethylcellulose, hydroxyethylcellulose, and hydroxypropylmethylcellulose, Ficolls (a polymeric sugar), hydroxyethylstarch (HES), dextrates (e.g., cyclodextrins, such as 2-hydroxypropyl-β-cyclodextrin and sulfobutylether-β-cyclodextrin), polyethylene glycols, and pectin. Other pharmaceutical carriers, excipients, optional ingredients and/or additives suitable for use in the compositions according to the present disclosure are listed in “Remington: The Science & Practice of Pharmacy”, 19.sup.th ed., Williams & Williams, (1995), and in the “Physician's Desk Reference”, 52.sup.nd ed., Medical Economics, Montvale, N.J. (1998), and in “Handbook of Pharmaceutical Excipients”, Third Ed., Ed. A. H. Kibbe, Pharmaceutical Press, 2000.

In some embodiments, the composition comprises at least one additional antimicrobial agent. In some embodiments, the antimicrobial agent is an antibacterial agent. Example antibacterial agents, include but are not limited to, aminoglycosides (e.g. amikacin, gentamicin, kanamycin, neomycin, netilmicin, tobramycin, paromomycin, streptomycin or spectinomycin); ansamycins (e.g. geldanamycin, herbimycin or rifaximin); carbacephems (e.g. loracarbef); carbapenems (e.g. ertapenem, doripenem, imipenem, meropenem); cephalosporins (e.g. cefadroxil, cefazolin, cefalexin, cefaclor, cefprozil, cefuroxime, cefixime, cefdinir, cefditoren, cefoperazone, cefotaxime, cefpodoxime, ceftazidime, ceftiaxone, cefepime, ceftaroline fosamil or ceftobiprole), fluoroquinolones (e.g. ofloxacin or pefloxacin), glycopeptides (e.g. teicoplanin, vancomycin, telavancin, dalbavancin or oritavancin); lincosamides (e.g. clindamycin or lincomycin); lipopeptides (e.g. daptomycin); macrolides (e.g. azithromycin, clarithromycin, erythromycin, roxithromycin, telithromycin, fidaxomicin or spiramycin); monobactams (e.g. aztreonam); nitrofurans (e.g. furzolidone or nitrofurantoin); oxazolidinones (e.g. linezolid, posizolid, radezolid or torezolid); penicillins (e.g. amoxicillin, ampicillin, azlocillin, dicloxacillin, flucloxacillin, mezlocillin, methicillin, nafcillin, oxacillin, penicillin (G or V), piperacillin, temocillin or ticarcillin); polypeptides (bacitracin, colistin, polymyxin B); quinolones (e.g. ciprofloxacin, enfloxacin, gatifloxacin, gemifloxacin, levofloxacin, lomefloxacin, moxifloxacin, nalidixic acid, norfloxacin, ofloxacin, trovafloxacin, grepafloxacin, sparfloxacin or temafloxacin); sulfonamides (e.g. mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim-sulfamethoxazole or sulfonamidochrysoidine); tetracyclines (e.g. demeclocycline, doxycycline, minocycline, oxytetracycline or tetracycline); and other antibacterial agents such as clofazimine, dapsone, capreomycin, cycloserine, ethambutol, ethionamide, isoniazid, pyrazinamide, rifampicin, rifabutin, rifapentine, streptomycin, arsphenamine, choramphenicol, fosfomycin, fusidic acid, metronidazole, mupirocin, platensimycin, quinupristin/dalfopristin, thiamphenicol, tigecycline, tinidazole or trimethoprim; or any combination thereof.

In some embodiments, the antimicrobial agent is an antifungal agent. Suitable antifungals include, but are not limited to, fluconazole, amphotericin B, nystatin, voriconazole, itraconazole, posaconazole and caspofungin or any combination thereof.

Typically, the concentration of antimicrobial particles present in the composition is sufficient to disrupt the biofilm (or part thereof) and/or render the microorganisms non-viable. In some embodiments, the concentration of antimicrobial particles is sufficient to promote disruption of the biofilm. In some embodiments, the concentration of antimicrobial particles is sufficient to render the microorganisms non-viable. The person skilled in the art would understand that the concentration of antimicrobial particles present in the composition will vary depending on the other ingredients present in the composition, the desired effect, the microorganism(s) being treated, the concentration of the microorganism being treated (i.e. microorganism numbers), the location of the microorganism being treated and the like. In some embodiments, the concentration of antimicrobial particles present in the composition is between 0.001 to 10 mg/mL, between 0.001 to 5 mg/ml, between 0.001 to 2 mg/ml, or between 0.001 to 1 mg/mL. In some embodiments, the concentration of antimicrobial particles present in the composition is at least about (in mg/mL) 0.001, 0.0025, 0.005, 0.0075, 0.01, 0.025, 0.05, 0.075, 0.1, 0.2. 0.25, 0.3, 0.4, 0.5, 0.6, 0.7, 0.75, 0.8, 0.9, 1, 2, 3, 4, 5, 6, 7, 8, 9. In some embodiments, the concentration of antimicrobial particles present in the composition is less than about (in mg/mL) 10, 9, 8, 7, 6, 5, 4, 3, 2, 1, 0.9, 0.8, 0.75, 0.7, 0.6, 0.5, 0.4, 0.3, 0.2, 0.1, 0.075, 0.05, 0.025, or 0.01. The concentration of antimicrobial particles present in the composition may also be provided in a range between any two of these upper and/or lower values. In some embodiments, the concentration of antimicrobial particles present in the composition is about 0.1 mg/mL.

Generally, the composition comprises the antimicrobial particle in an amount that is a therapeutically effective amount. In some embodiments, the therapeutically effective amount is provided by a single dose. In some embodiments, the therapeutically effective amount is provided by one or more doses administered as part of a course of treatment, for example, 1, 2, 3, 4, 5, 6, 7, 8, 9, 10, 11, 12, 13, 14, 15, 16, 17, 18, 19, 20, 21, 22, 23, 24, 25, 26, 27 or greater than 27 doses. The one or more doses may be administered on a daily, weekly or monthly basis. The one or more doses may be administered on a as needed basis.

Compositions that include the antimicrobial particles can be prepared for a variety of modes of administration and can be administered in a variety of unit dosage forms depending upon the end use and method of administration. In some embodiments, the composition is formulated as a wash solution, a dressing, or a wound gel. In further embodiments, the composition is formulated as tablets, pills, troches, capsules, aerosol spray, solutions, suspensions, gels, pastes, creams, or foams. In some embodiments, the composition is formulated for parenteral, e.g., intravenous, intradermal, subcutaneous, oral (e.g., inhalation), transdermal (topical), transmucosal, vaginal, topical and rectal administration.

The present disclosure provides compositions for both veterinary and human medical use. In some embodiments, there is provided a composition comprising one or more antimicrobial particles for use in a method of disrupting a biofilm. In some embodiments, there is provided a composition comprising one or more antimicrobial particles for use in treating a biofilm-related infection. In some embodiments, there is provided a composition comprising one or more antimicrobial particles when used in a method of disrupting a biofilm. In some embodiments, there is provided a composition comprising one or more antimicrobial particles when used in treating a biofilm-related infection.

While the composition has been described hereinabove with reference to both veterinary and human medical use, the person skilled in the art will appreciate that the particles and compositions described herein also have other uses. For example, the particles and compositions may be used to remove a biofilm off any surface contaminated with or suspected of contamination with a biofilm, for example, sensors, endoscopy equipment, optical fibres, machinery, capillaries, plants and the like. Accordingly, in some embodiments, the composition is formulated as a wash solution, coating solution, spray solution and the like.

In some embodiments, a composition that comprises the antimicrobial particles defined herein may have an antimicrobial characteristic (e.g., kills at least 70%, at least 80%, at least 90%, at least 95%, or at least 99% of the microorganisms (e.g., bacteria or fungi) present in the biofilm and/or reduces the amount of microorganisms that form the biofilm by at least 70%, at least 80%, at least 90%, at least 95%, or at least 99%, as compared to a similar biofilm without treatment.

### Methods and Uses

The inventors of the present application have surprisingly found that the antimicrobial particles as described herein can be used to disrupt biofilms or a part thereof. The present inventors have found that the antimicrobial particles can be “magnetically activated” by a magnetic field (e.g. rotating magnetic field), meaning that the antimicrobial particles according to at least some embodiments or examples can change shape, move, spin, vibrate and/or fragment when exposed to the magnetic field. Without wishing to be bound by theory, it is thought that “magnetic activation” of the particles imparts a physical force on the microbial cells in the biofilm resulting in disruption of the membrane and/or biofilm extracellular matrix, inactivating the pathogen. A schematic representation of this process is shown in FIG. 17. Significantly, the present inventors have found that use of both micro- and nano-particles provides further additional advantages such as improved anti-biofilm activity.

Accordingly, in another aspect there is provided a method of disrupting a biofilm. In some embodiments, the method comprises:

contacting the biofilm with the composition according to any embodiments or examples thereof as described herein; and

applying a magnetic field to the biofilm to magnetically activate the antimicrobial particles and thereby disrupt the biofilm.

In yet another aspect, there is provided use of the antimicrobial particles according to any embodiments or examples thereof as described herein for the disruption of a biofilm. In some embodiments, the use comprises contacting the biofilm with the composition as described herein; and

applying a magnetic field to the biofilm to magnetically activate the antimicrobial particles and thereby disrupt the biofilm.

In yet another aspect, there is provided use of an antimicrobial particle according to any embodiments or examples thereof as described herein in the manufacture of a medicament for treating a microbial infection or for disrupting a biofilm. In some embodiments, the biofilm is contacted with the medicament; and a magnetic field is applied to magnetically activate the antimicrobial particles and thereby disrupt the biofilm.

In yet another aspect, there is provided a method of treating a biofilm related disease in a subject. In some embodiments, the method comprises administering to the subject the composition according to any embodiments or examples thereof as described herein; and applying a magnetic field to the subject. In yet another aspect, there is provided use of the antimicrobial particles according to any embodiments or examples thereof as described herein for the treatment of a biofilm related disease in a subject. In some embodiments, the use comprises contacting the biofilm with the composition according to any embodiments or examples thereof as described herein; and

applying a magnetic field to the biofilm to magnetically activate the antimicrobial particles and thereby disrupt the biofilm.

In yet another aspect, there is provided use of an antimicrobial particle according to any embodiments or examples thereof as described herein in the manufacture of a medicament for the treatment of a biofilm related disease, disorder or infection in a subject. In some embodiments, the biofilm is contacted with the medicament; and a magnetic field is applied to magnetically activate the antimicrobial particles and thereby disrupt the biofilm.

In some embodiments of the methods and uses defined herein, the magnetic field is a rotating magnetic field, pulsed magnetic field or oscillating magnetic field. In some embodiments of the methods and uses defined herein, the magnetic field is a rotating magnetic field.

As used herein, the term “disrupting a biofilm” and variations thereof is defined broadly and includes one or more of the following: (i) disruption of the biofilm extracellular matrix; (ii) separation of one or more of the microorganisms forming the biofilm from the biofilm; (iii) rupture of the membrane of one or more of the microbes forming the biofilm; and (iv) lysis of one or more of the microbes forming the biofilm (for example, see FIG. 17).

As used herein, the term “contacting” refers to refers to bringing the biofilm or part thereof into physical contact with the antimicrobial particles under suitable conditions. The step of contacting the biofilm or part thereof with the antimicrobial particles may be carried out in any convenient or desired way. For example, if the contacting step is to be carried out on a medical device prior to use, the medical device may be immersed in a composition comprising the antimicrobial particles or the medical device may be flushed with a composition comprising the antimicrobial particles under appropriate conditions, for example at an appropriate concentration and for an appropriate length of time.

If the contacting step is to be carried out in vivo, the antimicrobial particle may be administered to a subject using a suitable administration route in a therapeutically effective amount. Alternatively, an implanted medical device may be coated with antimicrobial particles prior to implantation.

In some embodiments, the biofilm comprises bacteria. In some embodiments, the biofilm comprises Gram-positive bacteria, Gram-negative bacteria or a combination thereof. Importantly, in some embodiments, the antimicrobial particles described herein have broad spectrum antibacterial activity and have biocidal activity for both Gram-positive and Gram-negative bacteria. In some embodiments, the biofilm comprises bacteria of the genus Actinobacillus, Acinetobacter, Aeromonas, Bordetella, Brevibacillus, Brucella, Bacteroides, Burkholderia, Borelia, Bacillus, Campylobacter, Capnocytophaga, Cardiobacterium, Citrobacter, Clostridium, Chlamydia, Eikenella, Enterobacter, Escherichia, Entembacter, Francisella, Fusobacterium, Flavobacterium, Haemophilus, Helicobacter, Kingella, Klebsiella, Legionella, Listeria, Leptospirae, Moraxella, Morganella, Mycoplasma, Mycobacterium, Neisseria, Pasteurella, Proteus, Prevotella, Plesiomonas, Pseudomonas, Providencia, Rickettsia, Stenotrophomonas, Staphylococcus, Streptococcus, Streptomyces, Salmonella, Serratia, Shigella, Spirillum, Treponema, Veillonella, Vibrio, Yersinia, or Xanthomonas and combinations thereof. In some embodiments, the biofilm comprises bacteria of the genus Pseudomonas or Staphylococcus. In one embodiment, the biofilm comprises bacteria of the genus Pseudomonas. In one embodiment, the biofilm comprises bacteria of the genus Staphylococcus. In some embodiments, the bacteria is Escherichia coli, Pseudomonas aeruginosa, Staphylococcus aureus, Bacillus cereus, or combinations thereof. In some embodiments, the bacteria is Escherichia coli. In some embodiments, the bacteria is Pseudomonas aeruginosa. In some embodiments, the bacteria is Staphylococcus aureus. In some embodiments, the bacteria is Bacillus cereus.

In some embodiments, the biofilm comprises fungi. In some embodiments, the biofilm comprises fungi of the genus Cryptococcus, Aspergillus, Fusarium, Pneumocystis, Trichosporon, Blastoschizomyces, Malassezia, Saccharomyces, or Coccidioides and combinations thereof. In some embodiments, the biofilm comprises fungi of the genus Candida, Aspergillus, Cryptococcus, Trichosporon, Coccidioides, or Pneumocystis and combinations thereof. In some embodiments, the biofilm comprises fungi of the genus Candida, Cryptococcus or combinations thereof. In some embodiments, the fungi comprises Cryptococcus neoformans, Aspergillus fumigatus, Fusarium species, Pneumocystis species, Trichosporon asahii, Blastoschizomyces capitatus, Malassezia pachydermatis, Saccharomyces cerevisiae, or Coccidioides immitis or combinations thereof. In some embodiments, the fungi comprises Candida spp, including but not limited to C. albicans, C. glabrata, C. rugose, C. dubliniensis, C. parapsilosis, C. neoformans, C. krusei, or C. tropicalis. In some embodiments, the fungi comprise Candida albicans. In some embodiments, the fungi comprises Cryptococcus. In some embodiments, the fungi comprise Cryptococcus neoformans.

As would be understood by the person skilled in the art, microbes rarely exist as single-species planktonic forms. Most biofilms contain more than one microbial species (i.e. they are polymicrobial and may contain at least one bacterial species and/or at least one fungal species). In some embodiments, the biofilm comprises bacteria and fungi. In some embodiments, the bacteria and fungi are as described hereinabove. For example, the biofilm may comprise Staphylococcus aureus and Candida albicans. In another example, the biofilm may comprise P. aeruginosa and A. fumigatus.

The antimicrobial particles described herein can be magnetically activated by applying a magnetic field. As used herein, the term “magnetically activate” and variations thereof describes the use of a magnetic force(s) to cause motion and/or structural changes in the antimicrobial particles. For example, in some embodiments the magnetic field may cause the antimicrobial particles to change shape, move, spin, vibrate and/or fragment. In some embodiments the magnetic field may cause the antimicrobial particles to change shape, move, spin and/or vibrate. In some embodiments, the magnetic field may cause the antimicrobial particles to move, spin and/or vibrate. In some embodiments, the magnetic field may cause the antimicrobial particles to change shape. In some embodiments, the antimicrobial particles may become rod shaped, star shaped, spheroid shaped or a jagged sphere in response to a magnetic field.

Typically, the applied magnetic field is a rotating magnetic field (or partially rotating magnetic field) although any magnetic field with varying amplitude and/or direction (e.g. an oscillating magnetic field or a pulsed magnetic field) may also be suitable. As used herein, a “rotating magnetic field” is magnetic field that has moving polarities in which opposite poles rotate in space about some point or axis. In some embodiments, the rotational speed of the magnet is greater than (in rpm) 100, 200, 400, 600, 800, 1000, 1200, 1400, 1600, 1800, or 2000. In some embodiments, the rotational speed of the magnet is less than (in rpm) 3000, 2800, 2600, 2400, 2200, 2000, 1800, 1600, 1400, 1200, 1000, 800, 600, 400 or 200. The rotational speed of the magnet may also be provided in a range between any two of these upper and/or lower values. In some embodiments, the rotational speed of the magnet is between 100 rpm and 3000 rpm, between 500 rpm and 2500 rpm or between 1000 rpm and 2000 rpm. In some embodiments, the rotational speed of the magnet is between 500 rpm and 2000 rpm. In some embodiments, the rotational speed of the magnet is 1500 rpm.

The strength of the magnetic field (e.g. rotating magnetic field) is sufficient to magnetically activate the antimicrobial particles. In some embodiments, the magnetic field strength is at least (in mG) 250, 350, 450, 550, 650, 750, 850 or 950. In some embodiments, the magnetic field strength is less than (in mG) 1500, 1400, 1300, 1200, 1100, 1000, 900, or 800. The magnetic field strength may also be provided in a range between any two of these upper and/or lower values, although higher and lower magnetic strengths are also envisaged provided the strength of the magnetic field is sufficient to magnetically activate the antimicrobial particles. In some embodiments, the magnetic field strength is between 250 and 1500 mG, 350 and 1400 mG, 450 and 1300 mG, 550 and 1200 mG, 650 and 1100 mG or 750 and 1000 mG. In some embodiments, the magnetic field strength is about 250 mG, 350 mG, 450 mG, 550 mG, 650 mG, 750 mG, 775 mG, 800 mG, 850 mG, 950 mG, 1050 mG, 1150 mG, 1250 mG, 1350 mG, or 1450 mG. In some embodiments, the magnetic field strength is about 775 mG.

The source of the magnetic field (i.e. magnet or magnetic field generator) should be located at an appropriate distance from the biofilm, such that it is able to magnetically activate the antimicrobial particles and disrupt the biofilm. As would be appreciated by the person skilled in the art, the force experienced by an antimicrobial particle as a result of the magnetic field is inversely proportional to the distance from the magnet. In some embodiments, the source of the magnetic field located at least about (in mm) 1, 2, 3, 4, 5, 6, 7, 8, 9 or 10 from the biofilm. In some embodiments, the source of the magnetic field is located less than about (in mm) 50, 40, 30, 20, 10, 9, 8, 7, 6, 5, 4, 3, 2, or 1 from the biofilm. The distance may also be provided in a range between any two of these upper and/or lower values. In some embodiments, the source of the magnetic field is located 1 mm to 50 mm, 1 mm to 40 mm, 1 mm to 30 mm, 1 mm to 20 mm, 1 mm to 10 mm, or 1 mm to 5 mm from the biofilm. In some embodiments, the magnetic field is located about (in mm) 1, 2, 3, 4, 5, 6, 7, 8, 9 or 10 from the biofilm. In some embodiments, the magnetic field is located about 5 mm from the biofilm. As the person skilled in the art would appreciate, there are practical limitations which may limit the distance between the source of the magnetic field and the biofilm. In some embodiments, the source of the magnetic field should be located as close to the biofilm as is practical.

Typically, the magnetic field should be applied for a time sufficient to disrupt the biofilm or part thereof. In some embodiments, the magnetic field is applied for a predetermined period of time. In some embodiments, the magnetic field is applied for at least 5 minutes, at least 10 minutes, at least 20 minutes, at least 30 minutes, at least 60 minutes, at least 90 minutes or at least 120 minutes. In some embodiments, the magnetic field is applied for 1 minute, 2 minutes, 3 minutes, 4 minutes, 5 minutes, 10 minutes, 20 minutes, 30 minutes, 60 minutes, 90 minutes or 120 minutes. In some embodiments, the magnetic field is applied for a continuous period of time. In some embodiments, the magnetic field is applied periodically, for example for predetermined period of time followed by a break and then a further predetermined period of time.

In some embodiments, a biofilm is treated with an effective amount of the antibacterial particle. The phrase, an “effective amount”, as used herein, refers to an amount of antimicrobial particle that is sufficient to disrupt a biofilm or part thereof. Effective amounts may, for example, be determined by routine experimentation.

In some embodiments of the methods and uses herein, the biofilm is contacted with one or more additional antimicrobial agents. In some embodiments, the methods and uses further comprise contacting the biofilm simultaneously with one or more additional antimicrobial agents. In some embodiments, the methods and uses further comprise contacting the disrupted biofilm with one or more additional antimicrobial agents. Any suitable antimicrobial agent may be used, for example those defined herein. A suitable antimicrobial agent can be selected by the person skilled in the art, for example, based on knowledge of the microorganisms present in or predicted to be present in the biofilm.

Biofilms can form on living and non-living surfaces. The antimicrobial particles described herein are capable of disrupting biofilms on living and non-living surfaces. In some embodiments, the biofilm is formed on a living surface. In some embodiments, the biofilm is formed on a non-living surface. In some embodiments, the biofilm is formed on both a living and non-living surface. Non-limiting examples of living surfaces include wounds (e.g. open skin wounds, ulcers and the like), the urinary tract, lung, inner ear, oral cavity, teeth, plant tissue, and the like. Non-limiting examples of non-living surfaces include abiotic devices, cardiac implants, catheters, medical devices. In some embodiments, the antibacterial particles can be used to disrupt a biofilm on a hard to reach surface, for example the interior of a catheter, valve, mesh and the like. In addition, biofilm formation may occur without a surface. This is the case, for example, in cystic fibrosis-related lung infection where P. aeruginosa forms dense matrix-enclosed cell aggregates in the viscous mucus that are not attached to the epithelial cell lining. In some embodiments, the antimicrobial particles described herein are capable of disrupting biofilms that are not formed on a surface.

In some embodiments, the biofilm is located on or in a medical device or a portion thereof. In some embodiments, the medical device is selected from the group consisting of dentures, catheter, cannula, contact lens, a clamp, forcep, scissors, skin hook, tubing, needle, retractor, scaler, drill, chisel, rasp, saw, orthopedic device, orthopedic implants, optical fibre, dental implant, artificial heart valve, prosthetic joint, voice prosthetic, stent, implantable electronic devices, shunt, pacemaker, surgical pin, respirator, ventilator, and an endoscope and combinations thereof. In some embodiments, the particles and compositions described herein can be used to remove a biofilm from a device, such as those described herein. In some embodiments, the device is a catheter, cannula, endoscope, optical fibre or dental implant.

The antimicrobial particles described herein may also be used to treat a biofilm related disease in a subject. Biofilm related diseases are defined broadly and include biofilm related disorders. Biofilm related diseases include, but are not limited to, pneumonia, cystic fibrosis, periodontal disease, otitis media, chronic obstructive pulmonary disease, wound infection, oral infection, sinus infection, and a urinary tract infection and combinations thereof. In some embodiments, the biofilm related disease is a periodontal disease, such as gingivitis, periodontitis or breath malodor. In some embodiments, the biofilm-related disease is a wound infection. In some embodiments, the biofilm-related disease is an oral infection. In some embodiments, the biofilm related disease is acute and recurrent urinary tract infection, catheter-associated urinary tract infection, biliary tract infection, cystic fibrosis lung infection, chronic wound infection, catheter-associated urinary tract infection, chronic rhinosinusitis, chronic otitis media, contact lens-related keratitis, chronic osteomyelitis, chronic rhinosinusitis, endocarditis, chronic otitis media, orthopaedic implants, central venous catheter, orthopaedic implants, chronic osteomyelitis, colonization of nasopharynx, chronic rhinosinositis, chronic otitis media, chronic obstructive pulmonary disease, colonization of oral cavity and nasopharynx, recurrent tonsillitis or combinations thereof.

In some embodiments, the biofilm-related disease is a medical device-related infection. In some embodiments, the medical device is selected from the group consisting of dentures, catheter, contact lens, a clamp, forceps, scissors, skin hook, tubing, needle, retractor, scaler, drill, chisel, rasp, saw, catheter, orthopedic device, artificial heart valve, prosthetic joint, voice prosthetic, stent, shunt, pacemaker, surgical pin, respirator, ventilator, and an endoscope and combinations thereof. As would be understood by the person skilled in the art, the medical device should be suitable for use with a magnetic field.

In some embodiments, the antimicrobial particles can be disposed on a surface of a structure. In some embodiments, the structure can comprise those that may be exposed to microorganisms and/or that microorganisms can grow on such as, without limitation, metals, drug vials, medical instruments, medical implants, plastic devices and the like. In an embodiment, the structure can include textile articles, fibers, filters or filtration units (e.g., HEPA for air and water), plastic structures (e.g., made of a polymer or a polymer blend), glass or glass like structures on the surface of the structure, metals, metal alloys, or metal oxides structure, a structure (e.g., tile, stone, ceramic, marble, granite, or the like), and a combination thereof.

In some embodiments, after the antimicrobial particles is disposed on the surface, the structure may have an antimicrobial characteristic that is capable of killing a substantial portion of the microorganisms on the surface of the structure when exposed to a magnetic field (e.g. rotating magnetic field). The phrase “killing a substantial portion’ includes killing at least about 70%, at least about 80%, at least about 90%, at least about 95%, or at least about 99% of the microorganism (e.g., bacteria) on the surface that the antimicrobial particles are disposed on, relative to structure that does not have the antimicrobial particles disposed thereon.

In some embodiments, the method and uses described herein comprise administering to the subject the composition as defined herein; and applying a magnetic field (e.g. a rotating magnetic field) to the subject. As used herein, the term “administer” and “administering” are used to mean introducing the antimicrobial particles into a subject. When administration is for the purpose of treatment, the antibacterial particle may be provided before (e.g. coated on a device before implantation), at, or after the onset of, a symptom of a bacterial infection. The therapeutic administration of the antimicrobial serves to attenuate any symptom, or prevent additional symptoms from arising. When administration is for the purposes of treating a biofilm formed on an implanted device, in some embodiments the implanted device may be pre-coated with the antimicrobial particles using any technique known to the person skilled in the art.

Typically, when administration is for the purpose of treatment, the magnetic field is provided during, or after administration of the antimicrobial particle or composition comprising the antimicrobial particle. Any suitable magnetic field may be used. For example, the magnetic field may be provided by a magnetic resonance imaging device.

The antimicrobial particles may be administered by any suitable route. The route of administration may, for example, be targeted to the disease or disorder which the subject has and/or the site of biofilm formation. Examples include, but are not limited to, oral, topical, transdermal, intranasal, vaginal, rectal, intraarterial, intramuscular, intraosseous, intraperitoneal, epidural and intrathecal. In some embodiments, the antimicrobial particles are administered orally, intranasally, intravenously, intramuscularly, topically or intraperitoneally. In some embodiments, the antimicrobial particles may be administered orally. In some embodiments, the antimicrobial particles may be administered intranasally. In some embodiments, the antimicrobial particles may be administered intravenously. In some embodiments, the antimicrobial particles may be administered intramuscularly. In some embodiments, the antimicrobial particles may be administered intradermally. In some embodiments, the antimicrobial particles may be administered intraperitoneally. In some embodiments, the antimicrobial particles may be administered topically.

As used herein, the term “subject” refers to any organism that is susceptible to a biofilm. In some embodiments, the subject is a mammal, reptile, bird, insect or fish. In some embodiments, however, the subject is a mammal, particularly a primate, domestic animal, livestock or laboratory animal. In some embodiments, the subject is a human, a livestock animal (e.g., sheep, cow, horse, pig), a companion animal (e.g., dog, cat), or a laboratory animal (e.g., mouse, rabbit, rat, guinea pig, hamster). Example subjects include, but are not limited to, humans, monkeys, cats, koalas, dogs, horses, donkeys, sheep, pigs, goats, cows, mice, rats, rabbits, guinea pigs. In one embodiment, the subject is human. In one embodiment, the subject is a non-human mammal.

In some embodiments, a therapeutically effective amount of the antimicrobial particle is administered to a subject in need of treatment. As used herein, the term “therapeutically effective amount”, refers to the antimicrobial particle being administered in an amount sufficient to disrupt a biofilm or part thereof. The result can be the reduction and/or alleviation of the signs, symptoms, or causes of a disease or condition, or any other desired alteration of a biological system. In one embodiment, the term “therapeutically effective amount” refers to the antimicrobial particle being administered in an amount sufficient to result in a reduction of symptoms associated with a microbial biofilm infection. In some embodiments, a therapeutically effective amount refers to the amount of antimicrobial particle that is effective to disrupt a biofilm or part thereof without undue adverse side effects or to achieve a desired pharmacologic effect or therapeutic improvement with a reduced side effect profile. It is understood that “a therapeutically effective amount” can vary from subject to subject, due to variation in metabolism of the compound and any of age, weight, general condition of the subject, the condition being treated, the severity of the condition being treated, and the judgment of the prescribing physician. An appropriate “therapeutically effective amount” in any individual case may be determined by one of ordinary skill in the art using routine experimentation.

Without wishing to be bound by theory, it is thought that disruption of the biofilm by the antibacterial particles is a kinetically driven process (i.e. causes physical damage and/or removal) rather than involving a chemical reaction or interfering with cellular chemical processes. This is highlighted by (1) the lack of antibacterial behaviour when the post-magnetically treated particles were incubated with bacteria; and (2) the lack of antibacterial activity when intact biofilms were exposed to the magnetic field (see Table 1). Thus, in at least some embodiments, an advantage of the antimicrobial particles described herein is that they do not rely on the cells being metabolically active to have an antimicrobial effect. Therefore, in at least some embodiments, the antimicrobial particles described herein can inactivate metabolically active microbial cells, as well as “persister cells” which are metabolically dormant.

Studies have reported different methods of inducing mechano-responsive, force-induced membrane rupture, as a means of mitigating biofilm formation. For example, studies have highlighted the use of high-aspect-ratio nanostructures as a method of biofilm mitigation. Such technologies rely exclusively on passive antimicrobial action, where the surface passively interacts with the microbial species, and the cell-surface-adhesion processes induce cell death. Interestingly, reports of the efficacy of such nanostructured surface varies as a function of bacterial membrane rigidity. In general, the average biocidal activity is greater for Gram-negative bacteria than for Gram-positive cells, due to the inherent difference in their respective cell membrane structures: it is thought that Gram-negative bacterial cells are easier to rupture due to their relatively thin cell wall, and vice versa. Moreover, translation to industrially relevant surfaces has not thus far occurred, suggesting scalability challenges. The methods and uses at least according to some embodiments or examples as described herein may provide one or more advantages (or at least an alternative) over previously disclosed methods of inducing mechano-responsive, force-induced membrane rupture as a means of mitigating biofilm formation. These may include (but are not limited to) one or more of the following: 1) the antibacterial action may be similar for both Gram-negative and Gram-positive cells; 2) the antimicrobial effect may be scalable, for example more material can be used for larger biofilm systems; 3) Gallium based liquid metals may provide biocompatible materials; and/or 4) a high antimicrobial efficacy may be achieved.

Although the uses and methods have been described with emphasis on the disruption of biofilms in a therapeutic context, other (non-therapeutic) applications of the methods and uses are within the scope of this disclosure. For example, the particles or composition according to any embodiments or examples thereof as described herein may be used to disrupt biofilms on devices, machines or processing equipment, and in particular, biofilms formed on hard to reach surfaces (e.g. internal surfaces).

### Process

In yet another aspect, there is provided a process for forming a composition comprising antimicrobial particles, the process comprising:

(i) combining a liquid metal comprising gallium or an alloy thereof with magnetic iron particles to form a liquid metal ferrofluid, and

(ii) sonicating the liquid metal ferrofluid in an aqueous carrier fluid to form the antibacterial particles, wherein the antimicrobial particle comprises:

a liquid metal core comprising
a liquid gallium or alloy thereof, and
a plurality of magnetic iron particles, and

an inorganic passivation layer encapsulating the liquid metal core.

The liquid metal may be provided as discussed and described above. Generally, the liquid metal remains in liquid form in the presence of the aqueous carrier fluid. In some embodiments, the aqueous carrier fluid serves as a medium for distributing the liquid metal. In some embodiments, the aqueous carrier fluid serves as a medium for distributing the antimicrobial particles. For example, in some embodiments, the composition comprises a suspension of antimicrobial particles.

In some embodiments, the carrier fluid reacts with the liquid metal to form the inorganic passivation layer, as discussed above. For example, in some embodiments, the carrier fluid comprises an oxidizer. In some embodiments, the carrier fluid acts as an oxidizer. As used herein, the term “oxidizer” refers to a substance that yields oxygen that is available to bind with the liquid metal (or components of the liquid metal). Non-limiting examples of oxidizers include oxygen, air, ozone, hydrogen peroxide, and water. For example, in some embodiments, gallium oxide is formed from the reaction of gallium and oxygen. In some embodiments, gallium oxide hydroxide is formed from the reaction of gallium and oxygen in an aqueous environment.

In some embodiments, the inorganic passivation layer is a thin and self-limiting oxide shell, the thickness of the oxide layer may be increased by exposing the liquid metal to further oxidizing conditions. For example, heating the liquid metal in the presence of oxygen may increase the thickness of the metal oxide layer. Accordingly in some embodiments, the step (ii) comprises heating the liquid metal ferrofluid in an aqueous carrier fluid. However, the liquid metal ferrofluid should not be heated to a temperature which transforms the antimicrobial particle into a solid particle, for example rods of gallium oxide hydroxide. The liquid metal ferrofluid should not be heated to a temperature which de-alloys the liquid metal.

Generally no further processing is required and the composition comprising antimicrobial particles can be used as is formed from the original process. However, in some embodiments, the process further comprises removing at least a portion of the antimicrobial particles from the composition. For example, the antimicrobial particles may be separated from the suspension using any one of a number of devices and techniques known to those of ordinary skill in the art. Non-limiting examples of removal methods include settling, filtration, and centrifugation. The particles may then be further processed, depending on the desired application. Generally, the particles do not require purification based on size. Without wishing to be bound by theory, the present inventors have found that a composition comprising at least one microparticle and one nanoparticle provides improved microbicidal activity by disrupting the biofilm and lysing cells.

Any suitable method of combining the liquid metal with the magnetic iron particles to form a liquid metal ferrofluid can be used. In some embodiments, step (i) comprises grinding the liquid metal comprising gallium or an alloy thereof with magnetic iron particles under an inert atmosphere. As used herein, an “inert atmosphere” is one having an oxygen concentration at or below 0.3% (or 3000 ppm). In some embodiments, the oxygen concentration is at or less than 100 μm, for example between 1 ppm and 100 ppm or between 10 and 100 μm. In some embodiments step (i) is performed in a glove box purged with an inert gas. In some embodiments, the inert gas is argon or nitrogen. In some embodiments, grinding is carried out using a mortar and pestle.

In some embodiments, the concentrations and amounts of the components of the solution may influence one or more characteristics of the antimicrobial particles, such as their size and shape. In some embodiments, the mixing forces and other conditions, such as the ratio of the liquid metal, aqueous carrier fluid(s), magnetic iron particles to one another, and other considerations such as the process duration, temperature, and pressure, may each be adjusted to produce particles of different sizes and shapes.

In some embodiments, the liquid metal ferrofluid comprises magnetic iron particles in an amount (% w/w of fluid) of at least about 0.01, 0.05, 0.1, 0.5, 1, 2, 3, 4, 5, 6, 7, 8, 9 or 10. In some embodiments, the liquid metal ferrofluid comprises magnetic iron particles in an amount (% w/w of fluid) of less than about 10, 9, 8, 7, 6, 5, 4, 3, 2, 1, 0.5, or 0.1. In some embodiments, the liquid metal ferrofluid comprises magnetic iron particles in an amount (% w/w of fluid) between any two the these upper and/or lower values. For example, in some embodiments, the magnetic iron particles have a concentration of between about 0.1% w/w and 10% w/w, 0.1% w/w and 9% w/w, 0.1% w/w and 8% w/w, 0.1% w/w and 7% w/w, 0.1% w/w and 6% w/w, 0.1% w/w and 5% w/w, 0.1% w/w and 4% w/w, 0.1% w/w and 3% w/w, 0.1% w/w and 2% w/w, 0.1% w/w and 1% w/w, 1% w/w and 10% w/w, 2% w/w and 10% w/w, 3% w/w and 10% w/w, 4% w/w and 10% w/w, 5% w/w and 10% w/w, 6% w/w and 10% w/w, 7% w/w and 10% w/w, 8% w/w and 10% w/w, or 9% w/w and 10% w/w.

As would be appreciated by the person skilled in the art, step (i) can be used to reduce the particle size of the magnetic iron particles. For example, step (i) can reduce the diameter of the magnetic iron particles to less than about 1000 nm, less than about 900 nm, less than about 800 nm, less than about 700 nm, less than about 600 nm, less than about 500 nm, less than about 400 nm, less than about 300 nm, less than about 200 nm, less than about 100 nm, less than about 75 nm or less than about 50 nm. In one example, 90% of the magnetic iron particles pass through a 325-mesh sieve (i.e. 90% of particles smaller than 44 μm) prior to combining with the liquid metal.
A suitable carrier fluid can be provided by an aqueous carrier fluid. It will be appreciated that a suitable carrier fluid acts as a carrier for the antimicrobial particles (i.e. does not substantially dissolve the magnetic iron particles or the liquid metal). The carrier fluid can be selected to provide a stable liquid formulation comprising the antimicrobial particles as a suspension in the liquid formulation. The carrier fluid can also promote formation of the inorganic passivation layer. In some embodiments, the aqueous carrier fluid is water, for example ultrapure water. In some embodiments, the aqueous carrier fluid comprises salts, buffering agents or other additives. In some embodiments, the pH of the composition can be selected by the person skilled in the art to promote formation of the particles and/or formation of the inorganic passivation layer.

While step (ii) comprises sonicating the liquid metal ferrofluid in an aqueous carrier fluid to form the antibacterial particles, other means of applying a mixing force to produce form the antibacterial particles may be used. In accordance with various aspects, the mixing forces function to break up the liquid ferrofluid into particles. In accordance with some embodiments, the mixing forces are at least one of shear forces, cavitation forces, milling forces, ultrasonic forces, laser ablation forces, atomization forces, and compressive forces. One or more of these forces may be applied by at least one device. Non-limiting examples of the at least one device include high pressure homogenizers, jet stream devices, rotor-stator colloid mills, ball mills, high shear mixers, ultrasonic devices, mechanical alloying devices, laser devices, and atomization devices. As will be understood by a person skilled in the art, the mixing forces may be of any magnitude suitable for forming the antibacterial particles, as described herein.

Any suitable sonication device known to the person skilled in the art may be used. As the person killed in the art would appreciate certain variables can be adjusted when sonicating to achieve the desired outcome (i.e. antimicrobial particles as defined herein). The variables which can be adjusted in the application of sonication include, but are not limited to, the frequency of sonication applied, the power intensity at which the sonication is applied, the length of time the sonication is applied, the location of the transducer within the medium to be treated, and so forth.

The sonicating step is carried out for a period of time suitable for formation of the antimicrobial particles. A suitable period of time can be determined by the person skilled in the art using known techniques. In some embodiments, the mixing forces are applied for a period of time sufficient to produce a one or more antimicrobial particles comprising at least one of microparticle and at least one nano particle. In some embodiments, the sonicating is carried out for at least (in minutes) 1, 2, 5, 10, 15, 20 or 30. In some embodiments, the sonicating is carried out for between 5 minutes and 30 minute, for example 15 minutes. In some embodiments, the sonicating is carried out for 15 minutes or less, 10 minutes or less, 5 minutes or less, or 2 minutes or less. As used herein, “or less” requires sonication for at least 1 second.

In some embodiments, the sonicating is continuous. In alternative embodiments, the sonicating is pulsed.

In some embodiments, the sonicator has a probe tip diameter of 50 mm or less, 40 mm or less, 30 mm or less, 20 mm or less, 15 mm or less, 11 mm or less, 9 mm or less, 7 mm or less, 5 mm or less, 4 mm or less, 3 mm or less or 2 mm or less. As used herein, “or less” requires a probe tip diameter of greater than 0.1 mm. In some embodiments, the sonicating is carried out with a probe diameter of between 1 mm and 50 m, for example between 2 mm and 45 mm or between 3.7 mm and 41 mm.

Any suitable sonication intensity can be used. In some embodiments, the sonicating is carried out with an intensity of at least 1%, at least 5%, at least 10%, at least 20%, at least 30% at least 40% or at least 50%. In some embodiments, the sonicating is carried out with an intensity of between about 5% and 50%, about 5% and 40%, about 5% and 30%, about 5% and 20%, or about 5% and 15%. In some embodiments, the sonicating is carried out at with an intensity of 10%.

Any suitable sonication device displacement amplitude can be used. In some embodiments, the sonicating is carried out with sonication device displacement amplitude of between about 15 μm and 300 μm, or between about 40 μm and 300 μm, or between about 120 μm and 300 μm. In some embodiments, the sonicating is carried out with sonication device displacement amplitude of between about 15 μm and 300 μm, between about 15 μm and 120 μm, or between about 15 μm and 25 μm. In some embodiments, the sonicating is carried out at with sonication device displacement amplitude of about 40 μm.

Any suitable sonication frequency can be used. In some embodiments, the sonicating is carried out at a frequency of between about 60 Hz and about 60 kHz. In some embodiments, the sonicating is carried out at a frequency of between about 60 Hz and about 20 kHz. In some embodiments, the sonicating is carried out at a frequency of between about 20 kHz and about 60 kHz. In some embodiments, the sonicating is carried out at a frequency of between about 10 kHz and about 40 kHz. In some embodiments, the sonicating is carried out at a frequency of at least about (in kHz) 0.060, 0.5, 1, 2.5, 5, 7.5, 10, 12.5, 15, 17.5, 20, 22.5, 25, 27.5, 30, 32.5, 35, 37.5, 40, 42.5, 45, 47.5, 50, 52.5, 55 or 57.5. In some embodiments the sonicating is carried out at a frequency of less than about (in kHz) 60, 57.5, 55, 52.5, 50, 47.5, 45, 42.5, 40, 37.5, 35, 32.5, 20, 27.5, 25, 22.5, 20, 17.5, 15, 12.5, 10, 7.5, 5, 2.5, 1, 0.5, or 0.1. The sonicating frequency may also be provided in a range between any two of these upper and/or lower values. In some embodiments, the sonicating is carried out at a frequency of about 10 kHz. In some embodiments, the sonicating is carried out at a frequency of about 40 kHz.

Any suitable power consumption can be used. In some embodiments, the sonicating is carried out at a power consumption of less than about 280 watts, less than about 240 watts, less than about 200 watts, less than about 160 watts, less than about 120 watts or less than about 80 watts. As used herein, “or less” requires a power of greater than 0.1 watts. In preferred embodiments, the sonicating is carried out at a power of greater than 40 watts, greater than 60 watts, greater than 100 watts, greater than 140 watts, greater than 180 watts or greater than 220 watts. In some embodiments, the sonicating is carried out at a power of between 60 watts and 240 watts.

As would be understood by the person skilled in the art, the sonicating is carried out at a temperature which does not cause substantial loss of the liquid metal ferrofluid and/or aqueous carrier fluid. Accordingly, in some embodiments, the sonicating step is carried out at temperatures less than the boiling point of the aqueous carrier fluid. Typically, the sonicating step is carried out at temperatures above the melting point of the liquid metal. In some embodiments, the sonicating is carried out a temperature between about 15° C. and 100° C. In some embodiments, the sonicating is carried out at a temperature between about 20° C. and 50° C. In some embodiments, the sonicating is carried out at a temperature between about 25° C. and 40° C. In some embodiments, the sonicating is carried out a temperature less than about (in ° C.) 100, 90, 80, 70, 60, 50, 40, 30 or 25. In some embodiments, the sonicating is carried out a temperature greater than about (in ° C.) 15, 20, 25, 30, 35, 40 or 45. In some embodiments, the sonicating is carried out at ambient temperature. Where temperature control is required, the temperature can be maintained using any techniques known to the person skilled in the art (for example, by refrigeration or an ice bath).

As will be appreciated by the person skilled in the art sonicating parameters (including those mentioned above) can vary depending on sample volume, sonicator, sonicator probe, tip depth, sample vessel and the like. The person skilled in the art will be able to select the sonicating parameters required to form the antimicrobial particles defined herein.

### Kit

In another aspect, there is provided a kit comprising the antimicrobial particles or the composition according to any embodiments or examples thereof described herein. In another aspect, there is provided a kit comprising the liquid metal ferrofluid according to any embodiments or examples thereof described herein. In another aspect, there is provided a kit comprising liquid gallium or an alloy thereof and magnetic iron particles. In some embodiments, the kit comprises instructions for forming the antimicrobial particles described herein. In some embodiments, the kit comprises instructions for use of the antimicrobial particles described herein. The components of the kit can be used to form the particles or composition according to any embodiments or examples thereof as described herein.

### System

In another aspect, there is provided a system for disrupting a biofilm comprising the particles or composition according to any embodiments or examples thereof as described herein; and a magnetic field generator. In yet another aspect, there is provided a system for treating a biofilm related disease comprising the particles or composition according to any embodiments or examples thereof as described herein; and a magnetic field generator. In some embodiments, the system is also referred to as a magnetic treatment system.

In order to disrupt the biofilm and/or treat the biofilm related disease, the antimicrobial particles are magnetically activated. Therefore, the magnetic field generator is capable of generating a magnetic field capable of magnetically activating the antimicrobial particles. In some embodiments, the magnetic field generator is capable of generating an magnetic field that varies in amplitude and/or direction over time (e.g. either continuously or in a pulsed fashion). In some embodiments, the magnetic field generator is capable of generating an magnetic field that varies in direction over time. The change in field direction may be accomplished either by moving (e.g. rotating) the subject within a static magnetic field or by varying the field applied to the subject. The latter can be achieved, for example, by physically rotating the magnet hardware or by modulating currents in static electromagnet coils. In one embodiment, the magnetic field generator is capable of generating a pulsed magnetic field, i.e. the magnetic field is pulsed on and off. In one embodiment, the magnetic field generator is capable of generating an oscillating magnetic field. In one embodiment, the magnetic field generator is capable of generating a rotating magnetic field. Various magnetic field generators are feasible to generate such magnetic fields and are known to the person skilled in the art.

Any suitable magnetic field generator may be used. The examples of magnetic field generators given herein are for illustrative purposes. It is intended that this application covers a range of magnetic field generators, including small scale magnetic field generators that can target particular parts of the anatomy or devices and magnetic field generators that are large enough to treat the whole body or larger devices. The basic design principles of which will be apparent to those skilled in the art. As would be appreciated by the person skilled in the art, a whole body magnet does not have to treat the entire body simultaneously; it is acceptable to treat a portion at a time. This approach may reduce the cost and complexity of the magnetic field generator because the working region, over which the magnetic field has the required parameters, need only be large enough to encompass the largest anatomical structure, typically the abdomen. In some embodiments, the magnetic field generator is a hand held magnetic field generator. In some embodiments, the magnetic field generator is a portable or mobile magnetic field generator. In general, the magnetic field generator may share many common features with a magnet for magnetic resonance imaging. In some embodiments, the magnetic field generator comprises a MRI type magnet. In some embodiments, the magnetic field generator comprises a ferrite rare earth magnet. In some embodiments, the magnetic field generator comprises a neodymium magnet. In some embodiments, the magnetic field generator is capable of generating a rotating magnetic field, pulsed magnetic field or oscillating magnetic field as described herein. In some embodiments, the magnetic field generator is capable of generating a rotating magnetic field.

### Examples

The present disclosure is further described by the following examples. It is to be understood that the following description is for the purpose of describing particular embodiments only and is not intended to be limiting with respect to the above description.

### General Methods

XPS measurements. X-ray photoelectron spectroscopy (XPS) characterisation of the pre- and post-magnetised materials was performed using a Thermo K-Alpha instrument at a base pressure less than 1×10⁻⁹ mbar. The instrument utilises a monochromated Al Kα X-ray source to produce 1486.7 eV photons with an average spot size of 400 μm. A concentric hemispherical analyser (CHA) with a pass energy of 50 eV, in conjunction with a dwell time of 50 ms, was used to collect the emitted photoelectrons for all experiments. Typically, more than 70 scans in the specified energy ranges were performed and averaged. Core level binding energies (BEs) were referenced with respect to the adventitious C1s binding energy at 285.5 eV.

Bacterial strains, growth conditions, and sample preparation. Biofilms were grown from two strains of pathogenic bacteria, Pseudomonas aeruginosa ATCC 27853 and Staphylococcus aureus ATCC 25923. The bacterial strains were obtained from the American Type Culture Collection. These two species were chosen as representatives of Gram-negative and Gram-positive bacteria, respectively, which account for some of the most commonly occurring infection-related pathogens. They represent the two main morphologies among bacteria: rod and cocci cells, respectively. Furthermore, these bacterial species are disclosed to routinely cause post-operative infections.

For each experiment, bacteria cultures were grown on Luria-Bertani (LB) agar overnight at 37° C. Bacterial cells were collected from the culture via an inoculation loop and suspended in nutrient broth. These planktonic cell suspensions were grown overnight at 37° C. in 5 mL of LB broth from loop. The density of the bacterial suspensions was then adjusted to OD₆₀₀=0.1, after collection at the logarithmic stage of cell growth.

To obtain a mature biofilm, the planktonic cell suspensions were then added into individual glass-bottom Petri dishes (FluoroDish Cell Culture dishes, Part Number: FD35-100, World Precision Instruments, Sarasota, Fla., U.S.A.). Petri dishes were 35 mm in diameter with a 23 mm well, and were comprised of plastic walls with a glass-bottom, and were not pre-coated with any materials. The suspensions were allowed to grow for 24 h for S. aureus and 36 h for P. aeruginosa at 37° C. This approach allowed the bacteria to produce well-established biofilms.

SEM characterization. Scanning electron micrographs were obtained using a field-emission scanning electron microscope (FE-SEM). A FEI NOVA nano SEM (FEI company, Oregon, United States) at 5 kV and a ZEISS SUPRA 40 (VP, Oberkochen, BW, Germany) at 3 kV was used to image the systems using methods previously described. The resultant images were analyzed using a combination of the Gwyddion and Image J software suites. For cellular imaging, all samples were affixed, using 3% glutaraldehyde, and coated with a thin film of gold prior to imaging. The images were obtained from within the glass-bottom Petri dishes.

TEM characterization. High-resolution TEM (HRTEM) images were obtained with a JEOL 2100F microscope (JOEL, Musashino, Akishima, Tokyo, Japan) equipped with a Gatan Orius SC1000 CCD camera and operated at an acceleration voltage of 80 keV. Images were processed and analyzed using Digital Micrograph 2.31. For biological samples, aliquots were removed from the respective glass-bottom petri dishes and drop cast onto carbon mesh-coated TEM grids. The samples were allowed to dry prior to imaging.

AFM characterization. AFM images were obtained using a Cypher ES AFM (Oxford Instrument, Asylum Research, Santa Barbara, Calif., USA) under ambient conditions in air. Biolever BL-AC40TS cantilevers (Oxford Instrument, Asylum Research, Santa Barbara, Calif., USA, nominal spring constant kc=0.09 N/m) were used for all measurements, and imaging forces were minimized via a setpoint ratio (Imaging Amplitude (A)/free amplitude (A0)) of >0.7.

DLS characterization. Dynamic light scattering experiments were performed on an ALV-5022F light scattering spectrometer equipped with a laser wavelength of 633 nm. 100 μg/mL samples were suspended in water and measured in a cylindrical glass cuvette (inner diameter 8 mm) (LSI Instruments, Fribourg) held in a scattering vat at room temperature.

Confocal imaging and bacterial cell viability analysis. Confocal laser scanning microscopy (CLSM) (Fluoview FV1200 inverted micro-scope, Olympus, Tokyo, Japan) was used to evaluate the proportions of live and dead cells in each biofilm grown within a glass-bottom Petri dish. Cells within the biofilms grown in the glass-bottom Petri dishes were dyed using a LIVE/DEAD® BacLight™ Bacterial Viability Kit (including SYTO® 9 and propidium iodide (PI)) (Molecular Probes™ Invitrogen, Grand Island, N.Y., USA). Specifically, SYTO® 9 permeated both intact and damaged cell membranes, binding to nucleic acids and fluorescing green when excited by a 485 nm wavelength laser. PI dominantly entered cells that had undergone membrane damage, which are considered to be non-viable, and bound with higher affinity to nucleic acids than SYTO® 9. The non-viable cells will be permeated by propidium iodide.Bacterial suspensions were stained according to the manufacturer's protocol (Boulos, L., Prévost, M., Barbeau, B., Coallier, J. & Desjardins, R. LIVE/DEAD® BacLight™: Application of a New Rapid Staining Method for Direct Enumeration of Viable and Total Bacteria in Drinking Water. J. Microbiol. Methods 37, 77-86 (1999)). Discrepancies in viability assessment were avoided by ensuring that Syto® Green and propidium iodide fluorescence overlap was observed during image assessment. Furthermore, photobleaching of the SYTO® 9 dye was avoided by limiting each surface location to a single confocal scan. Live and dead cell ratio was quantified using Cell-C (https://sites.google.com/site/cellcsoftware/) providing a meaningful assessment of the antibacterial activity of the surface. Biomass of biofilm was quantified using COMSTAT version 2 (Heydorn, A. et al. Quantification of biofilm structures by the novel computer program COMSTAT. Microbiology 146, 2395-2407 (2000); Vorregaard, M. (Citeseer, 2008)).

### EXAMPLES

### Example 1—Preparation of Antimicrobial Particles

Galinstan was prepared in house by melting 68.5 wt % gallium (99.99%, Roto Metals Inc), 21.5 wt % indium (99.99%, Roto Metals Inc), and 10 wt % tin (99.9%, Roto Metals Inc) in a glass beaker at ˜250° C., using methods previously described (Tang, J., Zhao, X., Li, J., Zhou, Y. & Liu, J. Liquid metal phagocytosis: Intermetallic wetting induced particle internalization. Advanced Science 4, 1700024 (2017); Daeneke, T. et al. Liquid metals: fundamentals and applications in chemistry. Chemical Society Reviews 47, 4073-4111 (2018)). After heating, the resulting alloy was stirred and allowed to cool to room temperature, at which point it remained liquid. Freshly prepared galinstan was transferred into a nitrogen purged glove box (10 to 100 ppm O₂) for storage and further processing.

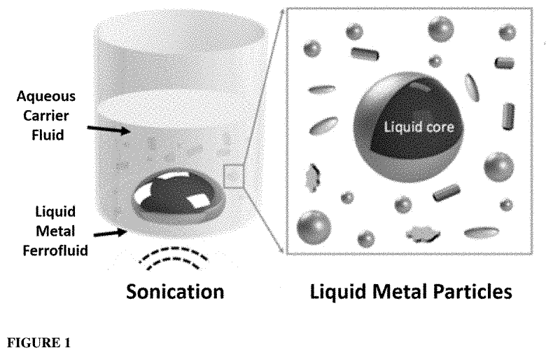

To prepare GLM-Fe particles, ˜10 g of galinstan and 1 wt % iron powder (Sigma Aldrich, -325 mesh, 97%) were ground in a mortar and pestle in a glove box for 30 min to achieve mechanical alloying (Zavabeti, A. et al. A liquid metal reaction environment for the room-temperature synthesis of atomically thin metal oxides. Science 358, 332-335 (2017); A. de Castro, I. et al. A Gallium-Based Magnetocaloric Liquid Metal Ferrofluid. Nano letters 17, 7831-7838 (2017)). The low solubility of Fe in the liquid metal ensured the formation of a biphasic alloy that contained magnetic iron particles, effectively forming a liquid metal-based ferrofluid. The resultant GLM-Fe mixture remained liquid at room temperature. The resultant GLM-Fe mixture was sonicated in Milli-Q (Merck Millipore) water (100 μg/mL) for 15 minutes. A schematic diagram of this process is shown in FIG. 1.

To prepare the GLM particles (i.e. particles without magnetic iron particles), the resultant galinstan was sonicated in Milli-Q (Merck Millipore) water (100 μg/mL) for 15 minutes.

### Example 2—Characterisation of Antimicrobial Particles

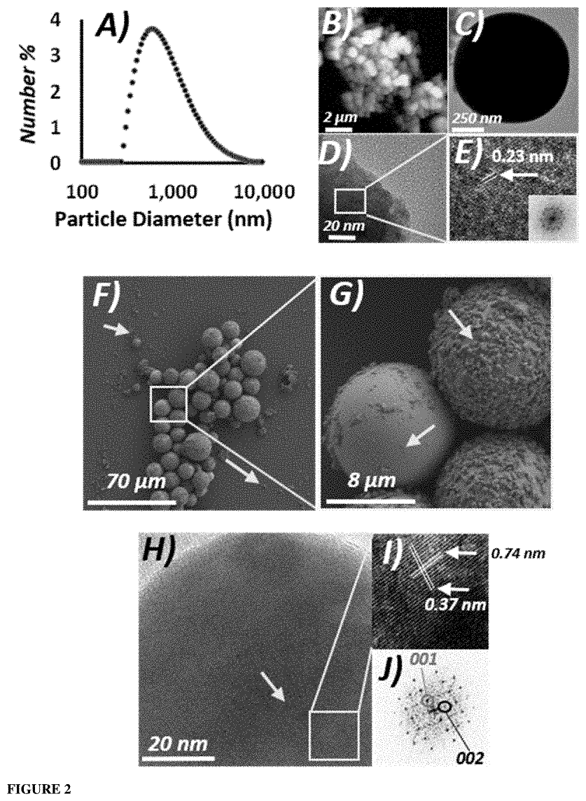

Scanning electron microscope (SEM) micrographs of the resulting GLM-Fe particles (FIGS. 2G and 2H) showed that the particles were predominantly contained a mixture of micro- and nano-sized spherical particles, with additional nano-fragments deposited on the silicon surface and attached to the periphery of the larger particles (highlighted by arrows in FIGS. 2G and 2H). The images highlighted the diverse morphologies of the antimicrobial particles.

Dynamic light scattering (DLS) data obtained for GLM-Fe particles in solution (FIG. 2A) revealed a large size distribution, with an average particle diameter ranging from ˜200 nm to ˜2 μm. The size range established via DLS measurements was commensurate with the information gleaned from SEM (FIGS. 2G and 2H), atomic force microscopy (AFM) (FIG. 2C), and transmission electron microscopy (TEM) (FIG. 2D), revealing a distribution of sizes.

Atomic resolution TEM images of the crystal lattice and the associated fast Fourier transform (FFT) analysis of the edges of the antimicrobial droplets revealed a dominant atomic lattice parameters of ˜0.24 nm (see FIGS. 2E and 2F). This value was commensurate with the orthorhombic [101] symmetry plane of gallium oxide hydroxide (GaOOH), suggesting that the material was oxidised to a greater degree following sonication. This indicated that the outer oxide layer comprises GaOOH. This was confirmed by TEM.

Encapsulation of iron particles within the Galinstan particles was visualised using HRTEM for a particle of the sonicated material (FIGS. 21 and 2J). Visual inspection of the HRTEM image showed a nanoparticle with a distinct, lower-contrast inclusions in the liquid metal center (for example, highlighted by the arrow in FIG. 21). Corresponding 2D-FFT analysis revealed that the inclusion displayed dominant atomic lattice parameters of 0.74 and 0.37 nm, a spacing commensurate with the 001 and 002 planes of orthorhombic Fe I, while the outer material consisted of galinstan (see FIGS. 2J and 2K). The physical size of the larger GLM-Fe particles precluded a similar analysis, but it is expected that the same encapsulation would remain for the larger particles.

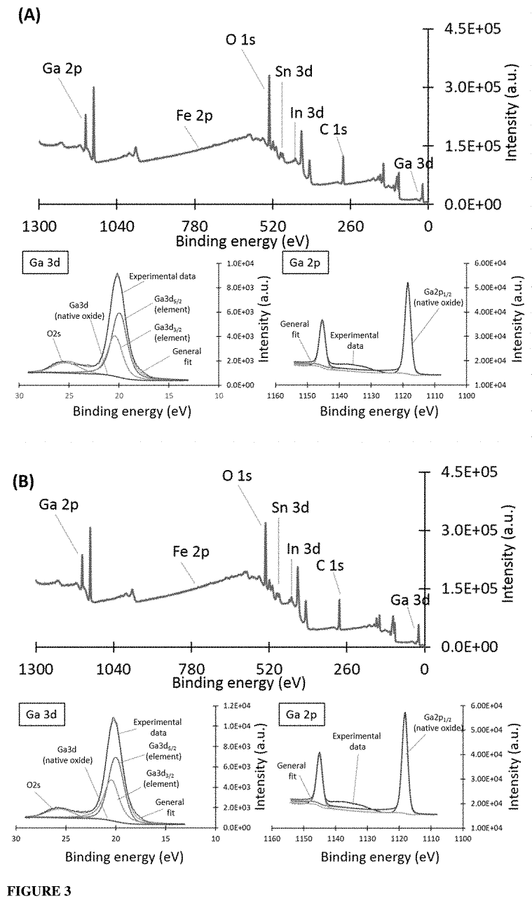

High resolution monochromated X-ray photoelectron spectroscopy (XPS) data pertaining to the sonicated material were collected and are shown in FIG. 3A. Peak positions and binding energy ranges were auto selected by the Avantage software. Peaks were assigned in accordance with the Avantage database, Ga⁰ peaks were located at 18.7 eV (Ga 3d), 159.5 eV (Ga 3s) and 1117 eV (Ga 2p) eV. In⁰ and Sn⁰ peaks were observed at 444 eV (In 3d) and 484.8 eV (Sn 3d), respectively. Oxygen and Carbon peaks were associated with the silicon substrate that was used as a support for the GLM-Fe particles.

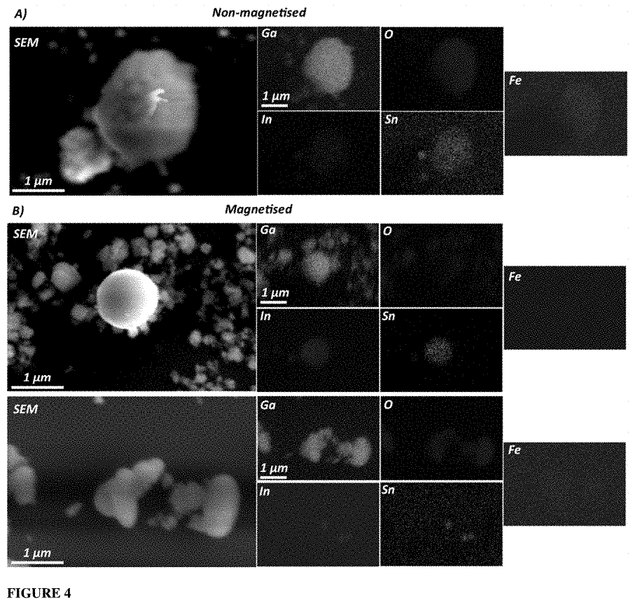

Energy-dispersive X-ray spectroscopy (EDX) data pertaining to the sonicated GLM-Fe mixture were collected and are shown in FIG. 4A. The respective SEM images shown alongside the EDX maps of Gallium (Ga), Indium (In), Oxygen (O), Tin (Sn), and Iron (Fe) (FIG. 4) confirming the presence of these elements in the GLM-Fe particles.

### Example 3—Magnetic Activation of Antimicrobial Particles

The GLM-Fe particles were exposed to a rotating ferrite rare-earth magnet, with a magnetic field strength of ˜775 milliGauss. The effect of an applied magnetic field on the GLM-Fe particles was assessed using SEM, TEM and DLS analyses, which revealed distinct differences between the pre- and post-magnetised particles.

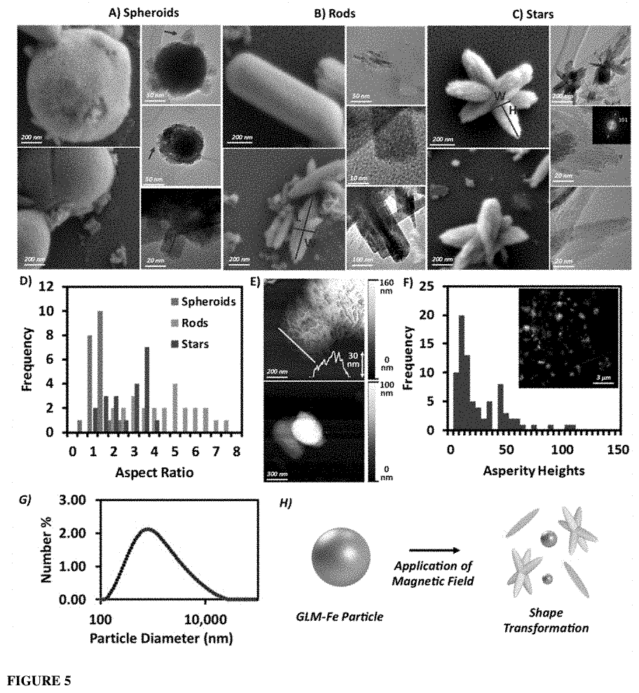

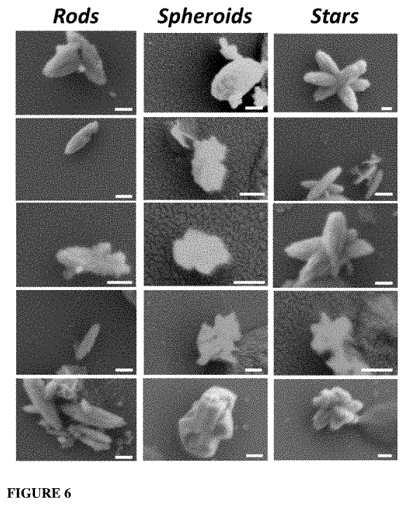

In response to magnetisation, GLM-Fe particles transformed from large spheres to smaller 3-dimensionally extruded particles (see FIGS. 5A, 5B, 5C and 5D). This indicated that the particles transform their shape in response to the magnetic field, into, for example, nanorods, nano-stars, and jagged-spheres (FIGS. 5A, 5B and 6). An extended collection of SEM images highlighted the resulting shapes adopted by the GLM-Fe (FIGS. 5B and 6), revealing the chaotic nature of the magnetically induced shape transformation on the particles.

The DLS data for the post-magnetised GLM-Fe particles (FIG. 5F) showed, in general, a widening of the size distribution following magnetisation, with significantly smaller particle sizes being observed. These data were commensurate with the particles undergoing magnetically induced fragmentation when compared to the data obtained for the pre-magnetised particles (FIG. 2A). The precise shape of these objects was found to be widely variable.

HRTEM imaging of a the edge of a magnetized GLM-Fe particle (FIG. 5C) revealed a nanosheet of material. The 2D-FFT analysis of the HRTEM image (FIG. 5C, inset) of the magnetically activated particle again revealed a dominant atomic lattice parameter of ˜0.24 nm [101] plane, suggesting that the outer layer of the particle comprises GaOOH. “Sharp” edges (i.e. asperities) were observed extending from magnetically activated GLM-Fe particles, and were found to be atomically thin (see FIG. 5D).

The XPS and EDX data for the magnetically activated GLM-Fe particles (FIGS. 3B and 4B, respectively) showed that no distinct differences could be noted between the pre-magnetised and post-magnetised samples, meaning that the magnetic field did not induce a measurable chemical change or de-alloying of the metallic constituents.

Together, this data suggested that the GLM-Fe materials transformed their shape in response to the externally-applied, rotating magnetic stimulus, but did not undergo a chemical transformation. This transformation may be rationalized by the liquid nature of the particles which may allow physical distortion of the particle under magnetic actuation. The increase in surface area due to de-formation could facilitate the oxidation of the surface. A schematic representation of magnetic activation of the GLM-Fe Particles is shown in FIG. 5E.

### Example 4—Treatment of Bacterial Biofilms Embedded with Antimicrobial Particles with Magnetic Field

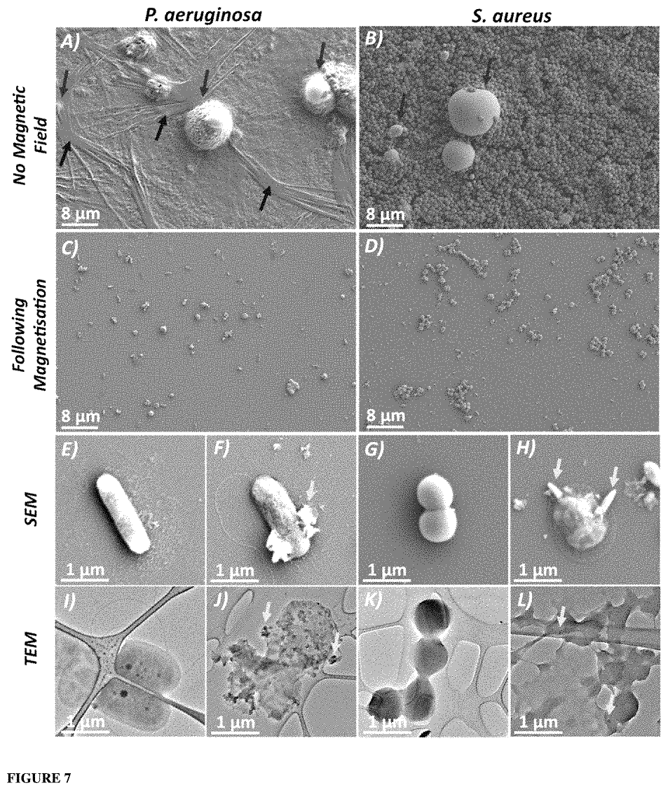

Mature P. aeruginosa and S. aureus biofilms were grown from a bacterial suspension in the presence of GLM-Fe particles (100 μg/mL). Representative low-magnification SEM images of the P. aeruginosa and S. aureus biofilms following growth in the presence of the GLM-Fe solution are shown in FIGS. 7A and 7B, respectively. Inspection of the images revealed the presence of thick, robust biofilms with the visible inclusion of the GLM-Fe particles (highlighted by the dark grey in FIGS. 7A and 7B). For the P. aeruginosa biofilm, thick extrusions of extracellular polymeric substances (EPS) were observed across the densely populated biofilm (highlighted by the black arrows in FIG. 7A), which is not as apparent for the S. aureus system. This is not unexpected, since P. aeruginosa cells are known to produce large volumes of EPS during biofilm formation (Myszka, K. & Czaczyk, K. Characterization of Adhesive Exopolysaccharide (EPS) Produced by Pseudomonas aeruginosa Under Starvation Conditions. Current Microbiology 58, 541-546 (2009)). Higher magnification SEM and TEM images of individual, isolated bacterial cells are shown in FIGS. 7E and 7I, and 7G and 7K, respectively, for the P. aeruginosa and S. aureus cells. This provided a baseline for a morphological analysis of the cells following magnetic exposure. Notably, the viable cells from both bacterial species appeared to be using the GLM-Fe particles as anchorage points. This suggested a lack of inherent bacterial toxicity for the GLM-Fe particles.

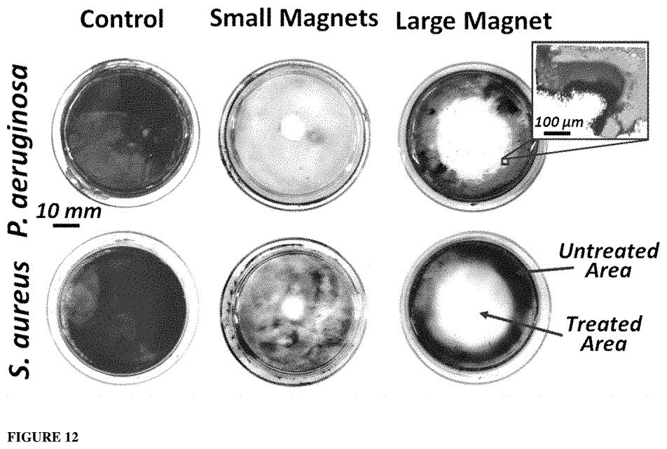

The biofilms were primarily exposed to a rotating ferrite rare-earth magnet, with a magnetic field strength of ˜775 milliGauss using the magnetic treatment system (FIGS. 8A and 8B). Where specified, a smaller neodymium magnet (FIG. 8C) was used, but only to show the effect of using a smaller magnetic field (see FIG. 12). Briefly, each Petri dish containing a bacterial biofilm was placed in the centre of the rotating magnetic field, atop the plastic stage for treatment. This brought the bottom of the sample to within 5 mm of the rotating magnet, which allowed magnetic activation of the magnetic particles. The rotational speed of the magnet was maintained at 1500 rpm for all experiments. Magnetic activation of the GLM-Fe particles caused visible vibration in the nutrient broth media and biofilm matrix.

The biofilms of both bacterial species were reassessed using low-magnification SEM following a 90 min magnetic exposure (FIGS. 7C and 7D) to visualise the effect of the magnetically activated GLM-Fe particles on the biofilms. The SEM images revealed the surfaces to be almost devoid of bacterial cells or cellular debris, while the remaining cells showed distinct signs of physical damage (FIGS. 7C and 7D). This showed that the magnetically activated GLM-Fe particles could disrupt a dense biofilm, while inducing cell death.

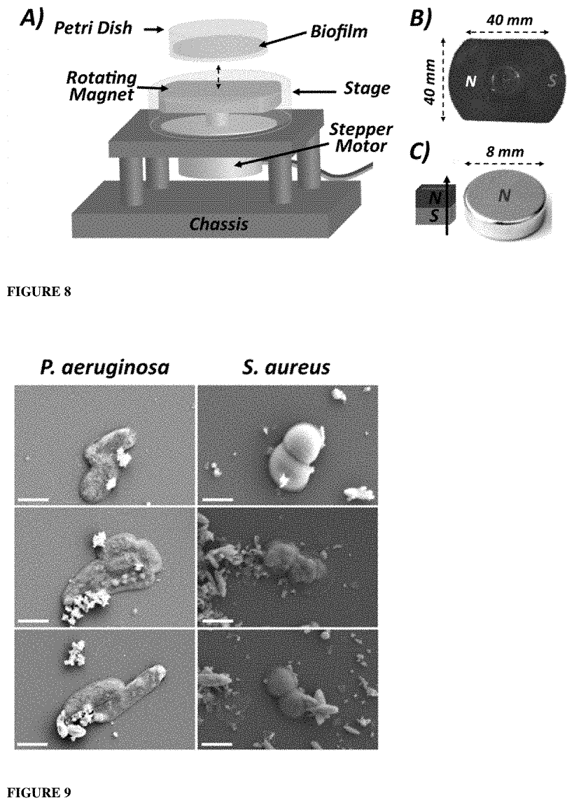

Higher magnification SEM and TEM images of individual P. aeruginosa and S. aureus cells following magnetic field exposure are shown in FIGS. 7F and 7J, and 7H and 7L, respectively. In these images, the bacterial cells of both pathogens appeared to be morphologically compromised when compared to the healthy cells (see FIGS. 7E and 7I, and 7G and 7K). The SEM images revealed the presence of damaged bacterial cells with small, metallic particles embedded within their membranes (highlighted by the white arrows). The magnetic particles exhibited the same morphologies observed for the magnetised particles in the absence of a biofilm (see FIGS. 5B and 6). Interestingly, for S. aureus cells (see FIG. 7H), it could be seen that certain regions of the cell's membrane appeared completely removed from the cell body. Additional SEM images of both species of bacteria following magnetisation are shown in FIG. 9, further highlighting the physical damage to the cellular membrane. This data suggested that the nanofragments of GLM-Fe particles were involved in the magnetically activated bacterial cell lysis. High resolution TEM images (FIGS. 7J and 7L) further highlighted the physical damage inflicted on the membrane of both bacterial species, with the images showing completely torn cells with fragments of GLM-Fe particles embedded throughout the intracellular spacing.

### Example 5—Treatment of Established Bacterial Biofilms with Antimicrobial Particles and a Magnetic Field

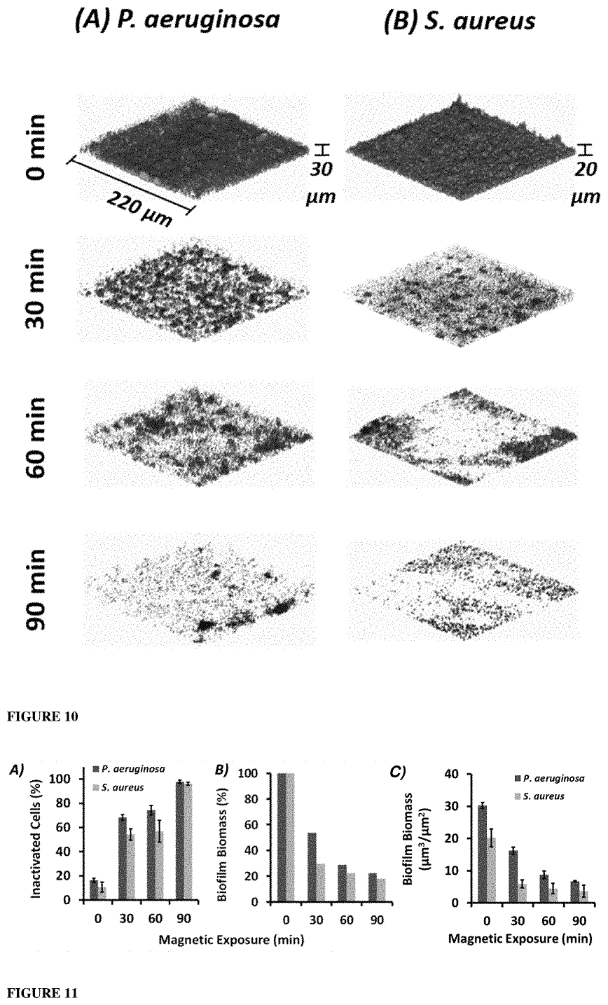

Electron microscopic investigations (see Example 4 and FIGS. 7, 9) showed that magneto-responsive GLM-Fe particles could disrupt an established biofilm. For practical application, it is imperative that the GLM-Fe particles can be simply placed in contact with a biofilm, and still be effective. To evaluate this application, in situ confocal scanning laser microscopy (CSLM) was employed to monitor the antibacterial efficacy of GLM-Fe particles as a function of magnetic field exposure time. Following growth in the absence of GLM-Fe particles, the initial viability of the cells within mature P. aeruginosa and S. aureus biofilms was assessed at the microscale via three-dimensional constructed CLSM images. The P. aeruginosa and S. aureus mature biofilms are shown in FIGS. 10E and 10I, respectively. Here, the viability of the bacterial cells within the biofilm could be quantitatively assessed via live versus dead fluorescent staining, shown in the CLSM images. The images revealed thick, active biofilms with a comparatively small number of non-viable cells being observed. These quantities were compared in the bar graph shown in FIG. 11A with 82% and 88% bacterial viability observed for P. aeruginosa and S. aureus, respectively. The corresponding biofilm biomass (raw biofilm mass; μm³/μm²) is shown in FIG. 11C, and is expressed as a percentage of initial biofilm mass in FIG. 11B. The levels of viable-to-inactivated cells were within the normal lifecycle and natural variation of cellular life within an established biofilm.

Following an initial assessment, the liquid covering the biofilm was removed and a 1 mL aliquot of GLM-Fe particle solution (100 μg/mL) was introduced to each biofilm. The resulting samples were exposed to a rotating magnetic field. Following 30 min of magnetic field exposure the cell viability and biofilm mass were reassessed using CLSM for both bacterial systems (see FIGS. 10F and 10J). Several distinct differences were noted in the CLSM images: 1) red pixels, which indicate non-viable bacterial cells, were now clearly seen in both images, 2) the thickness of the biofilm had decreased, and 3) regions devoid of biofilm (white areas) could now be seen.

This trend continued at time increments of 60 min (see FIGS. 10G and 10K) and 90 min (see FIGS. 10H and 10L). Importantly, after 90 min of exposure to the magnetic field ˜99% of both P. aeruginosa and S. aureus cells were inactivated, indicating almost complete cell death in the pathogenic community (see FIG. 11). The data indicated that the biofilms exposed to GLM-Fe particles in the presence of a rotating magnetic field experienced both bacterial cell lysis and a drastic reduction in the biofilm mass. To the best of the inventors' knowledge, this is the first reported method for substantial biofilm disintegration, in addition to antibacterial efficacy. A review of the literature has suggested that the GLM-Fe nanoparticle treatment described here appears to be the only therapeutic method that is capable of initiating bacterial cell lysis and biofilm disintegration substantially simultaneously.

To visualise the range of the magnetically induced antibacterial behaviour, crystal violet staining was conducted on untreated, and magnetically treated P. aeruginosa and S. aureus biofilms, where the size of the magnet, and hence magnetic field, was altered (see FIGS. 8B, 8C, and 12). Distinct voids in the biofilm treated with GLM-Fe particles under magnetic stimulation could be noted following 90 minutes of treatment. Importantly, the size of the void increased when a larger magnet was utilised (see FIGS. 8B and 12), and vice versa when a smaller magnet was used (see FIGS. 8C and 12). This revealed a localised, directionally proportionate anti-biofilm action of the GLM-Fe particles following exposure to the rotating magnetic field i.e. the treatment area could be readily controlled by adjusting the magnitude of the magnetic field, which could be localised to the targeted area.

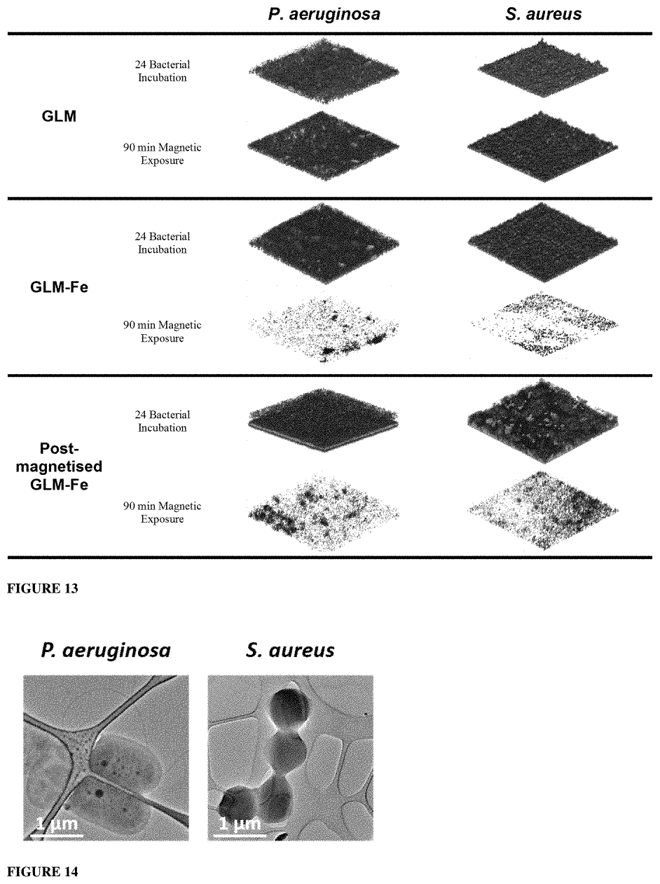

To confirm that the observed antibacterial behaviour was not chemically induced (via metal ion leaching or another chemical mechanism), 100 μg/mL solutions of GLM-Fe and pre-magnetised GLM-Fe particles were separately incubated with P. aeruginosa and S. aureus biofilms for 24 hours, without exposure to a magnetic field. Additionally, 100 μg/mL solutions of GLM particles, which did not contain magnetic iron inclusions, were also placed in contact with biofilms of both species, and placed under the rotating magnetic field. The resulting bacterial viability of both the P. aeruginosa and S. aureus biofilms was then assessed for all systems. In all cases, a thriving biofilm was observed. Representative CLSM images are shown in FIG. 13, and the results are tabulated in Table 1. Importantly, these experiments revealed that the particles were not toxic to the growth of bacteria in their pre- and post-magnetised forms. The TEM images of both species of bacteria co-cultured with non-magnetised GLM-Fe particles for 24 hours showed no signs of cellular particle uptake (FIG. 14).

TABLE 1
Assessment of Particle-Induced Bacterial Toxicity

| | | Biofilm | Antibacterial |
|---|---|---|---|
| Material | Condition | Degradation | Behaviour |
| GLM | 24 Bacterial | x | x |
| | Incubation | | |
| | 90 min Magnetic | x | x |
| | Exposure | | |
| GLM-Fe | 24 Bacterial | x | x |
| | Incubation | | |
| | 90 min Magnetic | ✓ | ✓ |
| | Exposure | | |
| Post- | 24 Bacterial | x | x |
| Magnetised | Incubation | | |
| GLM-Fe | 90 min Magnetic | ✓ | ✓ |
| | Exposure | | |

✓: Positive;
x: Negative

### Example 6—Treatment of Established Fungal Biofilms with Antimicrobial Particles and a Magnetic Field

Mature Candida albicans and Cryptococcus neoformans biofilms were grown from fungal suspensions. Suspensions of GLM-Fe particles (100 μg/mL) were added into fungal biofilms. GLM-Fe particles were activated via rotating magnetic fields. Biofilms were observed using confocal laser scanning microscopy after the treatment. It was shown that biofilm was removed and destroyed by the magnetically treated GLM-Fe particles (Data not shown).

### Example 7—Magnetically Activated Antimicrobial Particles are not Cytotoxic for Eukaryotic Cells

Cytotoxicity of GLM and GLM-Fe (pre- and post-magnetised) particles against a eukaryotic cell line, specifically human embryonic kidney (HEK) cells, was assessed using the AlamarBlue assay (S. N. Rampersad, Sensors 2012, 12(9), 12347-12360. HEK293 cells (ATCC) were seeded onto 96-well plates at a cell density of 15,000 cells per well in 100 μL completed media (Dulbecco's Modified Eagle's Medium-high glucose (DMEM, Sigma-Aldrich) supplemented with 10% (v/v) Fetal bovine serum (FBS-Sigma-Aldrich) and 1% penicillin/streptomycin (Life Technologies)). The cells were then incubated at 37° C., 5% CO₂ for 24 h. Subsequently, the medium was removed and replenished with 100 μL of completed medium containing the relevant particle at different concentrations. To evaluate the impact of magnetic activation of HEK cells in the presence of GLM-Fe, the cells were seeded into glass-bottom Petri dishes, to mimic the conditions of the treatment, and then exposed to a rotating rare-earth magnet, with a magnetic field strength of ˜775 milliGauss which were then treated with the same conditions as the non-magnetised plates. After 48 hours of incubation, the cells were washed twice with Dulbecco's Phosphate Buffered Saline solution (DPBS, Sigma Aldrich) before being incubated with fresh media for 2 hours at 37° C., 5% CO₂. Fluorescence was measured at an excitation wavelength of 530 nm and an emission wavelength of 590 nm using a NovoStar microplate reader. The experiments were performed in triplicate, and relative cell viability was calculated as the percentage viable compared to control cells in completed media without the addition of antimicrobial particles. Positive controls were established using SDS and Triton X-100 (0.1 wt %/vol) to show the efficacy of the AlamarBlue assay. This treatment induces cell death in the HEK cells, meaning that it validates the viability assay.

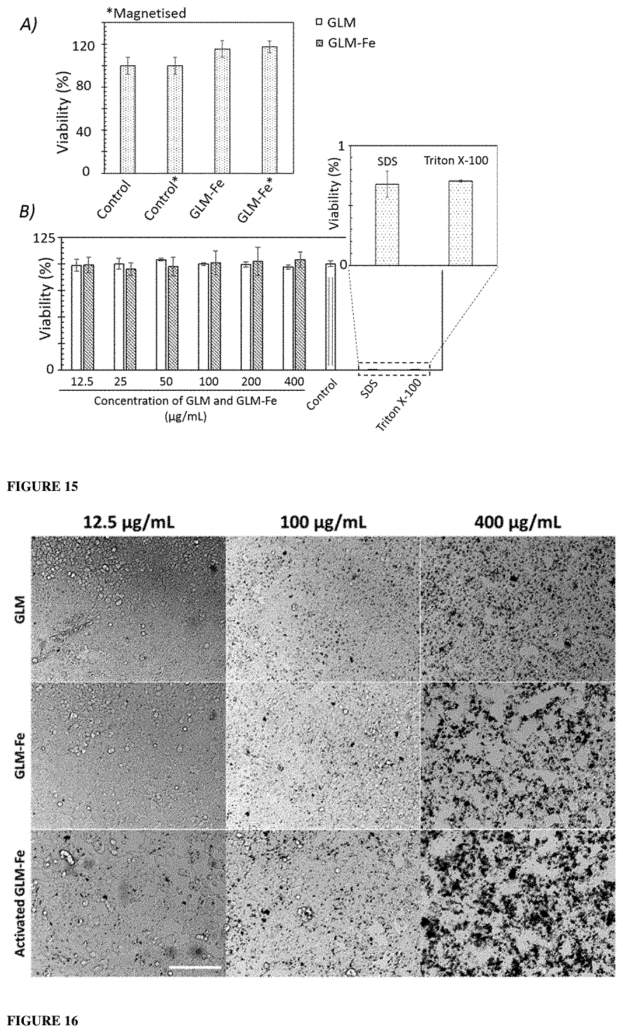

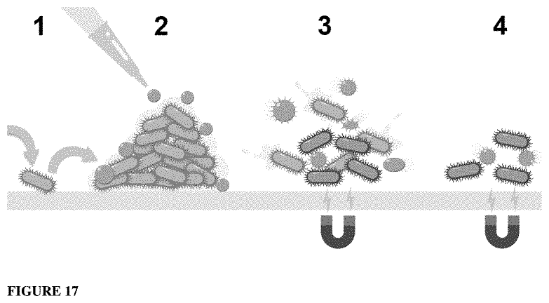

As shown in FIG. 15, the cell viability data suggested that GLM or GLM-Fe particles were well-tolerated by HEK cells, even on incubation with particle concentrations up to 400 μg/mL (see FIG. 15B). When exposed to a 750 milliGauss rotating magnetic field, GLM-Fe particles did not impose any toxicity or physical damage to the HEK cells (see FIG. 15A). FIG. 16 shows the corresponding optical micrographs of the HEK cells in solution with varying concentrations of liquid metal particles. Here, the images illustrate that after treatment with the respective particles, the cells were well-spread over the surfaces in a confluent manner. It was noted that under both experimental conditions (with and without exposure to magnetic field) and at high concentration (i.e. 400 μg/mL), the cells formed self-organised clusters, leading to the creation of bigger spaces between the cell clusters compared to those treated with lower nanoparticle concentrations. These results suggested that the particles physically engaged with the cells, but did not cause cell death.

In combination, the results indicated that magnetized GLM-Fe particles displayed advantageous and unexpected differential cell lysis properties i.e. treatment with the liquid metal particles and magnetic field could inactivate bacterial cells, but did not induce any significant physical damage or cytotoxicity to mammalian cells. Without wishing to be bound by theory, it is thought that eukaryotic cells were capable of distorting in response to the movement of magnetically activated GLM-Fe particles without damage to the cells' membrane or the internal cytosolic material within the cells.

The advantageous and unexpected results of this work showed antimicrobial particles described and exemplified herein can actively disrupt biofilms and/or deactivate individual pathogens. This work provides a promising method for the treatment of biofilm related diseases, for example infected wounds, as well as for medical and industry applications where biofilms are a significant challenge.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

The present disclosure is further defined by the following numbered paragraphs:
1. An antimicrobial particle comprising:
a liquid metal core comprising
   a liquid gallium or alloy thereof, and
   a plurality of magnetic iron particles; and
an inorganic passivation layer encapsulating the liquid metal core.
   2. The antimicrobial particle of paragraph 1, wherein the particle is a microparticle or a nanoparticle.
   3. The antimicrobial particle of paragraph 1 or paragraph 2, wherein the liquid gallium alloy comprises gallium and one or more metals selected from the group consisting of indium, tin, zinc, aluminium and copper.
   4. The antimicrobial particle of any one of paragraphs 1 to 3, wherein the liquid gallium or alloy thereof comprises an alloy of gallium and indium or an alloy of gallium, indium and tin.
   5. The antimicrobial particle of any one of paragraphs 1 to 4, wherein the liquid gallium or alloy thereof comprises eGaIn or Galinstan.
   6. The antimicrobial particle of any one of paragraphs 1 to 5, wherein the liquid gallium or alloy thereof consists of gallium.
   7. The antimicrobial particle of any one of paragraphs 1 to 6, wherein the magnetic iron particles comprise Fe, Fe₃O₄, Fe₂O₃, γ-Fe₂O₃, or combinations thereof.
   8. The antimicrobial particle of any one of paragraphs 1 to 7, wherein the magnetic iron particles comprise orthorhombic Fe I.
   9. The antimicrobial particle of any one of paragraphs 1 to 8, wherein the magnetic iron particles are nanoparticles.
   10. The antimicrobial particle of any one of paragraphs 1 to 9, wherein the magnetic iron particles have an average diameter of 35 nm to 1000 nm.
   11. The antimicrobial particle of any one of paragraphs 1 to 10, wherein the magnetic iron particles have a concentration of between 0.1% w/w and 10% w/w.
   12. The antimicrobial particle of any one of paragraphs 1 to 11, wherein the inorganic passivation layer comprises a metal oxide or a metal sub-oxide or a combination thereof.
   13. The antimicrobial particle of any one of paragraphs 1 to 12, wherein the inorganic passivation layer comprises gallium oxide hydroxide (GaOOH) or gallium oxide (Ga₂O₃) or a combination thereof.
   14. The antimicrobial particle of paragraph 13, wherein the inorganic passivation layer comprises at least 90% gallium oxide GaOOH.
   15. The antimicrobial particle of any one of paragraphs 1 to 14, wherein the inorganic passivation layer has a thickness of between 0.7 and 1.4 nm.
   16. The antimicrobial particle of any one of paragraphs 1 to 15, having an average diameter of between 80 nm to 10 μm.
   17. The antimicrobial particle of any one of paragraphs 1 to 16, wherein the particle is a sphere.
   18. The antimicrobial particle of any one of paragraphs 1 to 17, wherein in response to a rotating magnetic field the particle is capable of becoming rod shaped, star shaped, spheroid shaped or a jagged sphere.
   19. The antimicrobial particle of any one of paragraphs 1 to 18, wherein in response to a rotating magnetic field the particle is capable of fragmenting.
   20. A composition comprising one or more antimicrobial particles according to any one of paragraphs 1 to 19 and a carrier fluid.
   21. The composition of paragraph 20, comprising at least one microparticle and at least one nanoparticle.
   22. The composition of paragraph 20 or paragraph 21, wherein the carrier fluid is a pharmaceutically acceptable carrier fluid or a biocompatible carrier fluid.
   23. The composition of any one of paragraphs 20 to 22, wherein the carrier fluid is water.
   24. The composition of any one of paragraphs 20 to 23, comprising at least one additional antimicrobial agent.
   25. The composition of any one of paragraphs 20 to 24, wherein the concentration of the antimicrobial particles is between 1 μg/mL and 1 mg/mL.
   26. A method of disrupting a biofilm, the method comprising:
contacting the biofilm with the composition according to any one of paragraphs 20 to 25; and
applying a magnetic field to the biofilm to magnetically activate the antibacterial particles and thereby disrupt the biofilm.
   27. The method of paragraph 26, wherein the magnetic field is a rotating magnetic field.
   28. The method of paragraph 27, wherein the rotating magnetic field strength is between 250 and 1500 milliGuass.
   29. The method of paragraph 27 or paragraph 28, wherein the rotational speed of the magnet is between 500 rpm and 2000 rpm.
   30. The method of any one of paragraphs 26 to 29, wherein the magnetic field is located within 1 mm to 50 mm of the biofilm.
   31. The method of any one of paragraphs 26 to 30, wherein the magnetic field is applied for at least 5 minutes, at least 10 minutes, at least 20 minutes, at least 30 minutes, at least 60 minutes, at least 90 minutes or at least 120 minutes.
   32. The method of any one of paragraphs 26 to 31, further comprising contacting the biofilm simultaneously with an additional antimicrobial agent.
   33. The method of any one of paragraphs 26 to 32, further comprising contacting the disrupted biofilm with an additional antimicrobial agent.
   34. The method of any one of paragraphs 26 to 33, wherein the biofilm is located on or in a medical device or portion thereof.
   35. The method of any one of paragraphs 26 to 34, wherein the biofilm is formed from bacteria and/or fungi.
   36. The method of paragraph 35, wherein the biofilm is formed from bacteria of the genus Actinobacillus, Acinetobacter, Aeromonas, Bordetella, Brevibacillus, Brucella, Bacteroides, Burkholderia, Borelia, Bacillus, Campylobacter, Capnocytophaga, Cardiobacterium, Citrobacter, Clostridium, Chlamydia, Eikenella, Enterobacter, Escherichia, Entembacter, Francisella, Fusobacterium, Flavobacterium, Haemophilus, Helicobacter, Kingella, Klebsiella, Legionella, Listeria, Leptospirae, Moraxella, Morganella, Mycoplasma, Mycobacterium, Neisseria, Pasteurella, Proteus, Prevotella, Plesiomonas, Pseudomonas, Providencia, Rickettsia, Stenotrophomonas, Staphylococcus, Streptococcus, Streptomyces, Salmonella, Serratia, Shigella, Spirillum, Treponema, Veillonella, Vibrio, Yersinia, or Xanthomonas.
   37. The method of paragraph 36, wherein the bacteria is Escherichia coli, Pseudomonas aeruginosa, Staphylococcus aureus, Bacillus cereus, or combinations thereof.
   38. The method of paragraph 35, wherein the biofilm is formed from fungi of the genus Candida, Aspergillus, Cryptococcus, Trichosporon, Coccidioides, or Pneumocystis.
   39. The method of paragraph 38, wherein the fungi is Candida, Crytococcus or combinations thereof.
   40. A process for forming a composition comprising antimicrobial particles, the process comprising:
(i) combining a liquid metal comprising gallium or an alloy thereof with magnetic iron particles to form a liquid metal ferrofluid, and
(ii) sonicating the liquid metal ferrofluid in an aqueous carrier fluid to form the antibacterial particles, wherein the antimicrobial particle comprises
a liquid metal core comprising
   a liquid gallium or alloy thereof, and
   a plurality of magnetic iron particles, and an inorganic passivation layer encapsulating the liquid metal core.
      41. The process of paragraph 40, wherein step (i) comprises grinding the liquid metal comprising gallium or an alloy thereof with magnetic iron particles under an inert atmosphere.
      42. The process of paragraph 40 or paragraph 41, wherein the grinding is carried out using a mortar and pestle.
      43. The process of any one of paragraphs 40 to 42, wherein the liquid metal ferrofluid comprises 0.1% w/w to 10% w/w magnetic iron particles.
      44. The process of any one of claims 40 to 43, wherein the aqueous carrier fluid is water.
      45. The process of any one of paragraphs 40 to 44, wherein the sonicating is carried out for between 5 minutes and 30 minutes.
      46. The process of any one of paragraphs 40 to 45, wherein the sonicating is carried out at a temperature less than 40° C., or less than 30° C., or less than 25° C.
      47. The process of any one of paragraphs 40 to 46, wherein the sonicating is carried out at a frequency of between 60 Hz and 60 kHz.
      48. The process of any one of paragraphs 40 to 47, wherein the sonicating is carried out with sonication intensity of about 10%.
      49. The process of any one of paragraphs 40 to 48, wherein the sonicating is carried out with a probe diameter of between 3.7 mm to 41 mm.
      50. The process of any one of paragraphs 40 to 49, wherein the sonicating is carried out at a power of between 60 watts and 240 watts.
      51. A method of treating a biofilm related disease in a subject, the method comprising administering to the subject an antimicrobial particle according to any one of paragraphs 1 to 19 or the composition according to any one of paragraphs 20 to 25; and applying a magnetic field to the subject.

Having thus described in detail various embodiments of the present disclosure, it is to be understood that the present disclosure defined by the above numbered paragraphs is not to be limited to particular details set forth in the above description as many apparent variations thereof are possible without departing from the spirit or scope of the present disclosure.

1: An antimicrobial particle comprising:
a liquid metal core comprising
a liquid gallium or alloy thereof, and
a plurality of magnetic iron particles; and
an inorganic passivation layer encapsulating the liquid metal core.

2: The antimicrobial particle of claim 1, wherein the particle is a microparticle or a nanoparticle.

3: The antimicrobial particle of claim 1, wherein the liquid gallium or alloy thereof comprises an alloy of gallium and indium or an alloy of gallium, indium and tin or consists of gallium.

4: The antimicrobial particle of claim 1, wherein the magnetic iron particles comprise Fe, Fe₃O₄, Fe₂O₃, γ-Fe₂O₃, or combinations thereof.

5: The antimicrobial particle of claim 1, wherein the magnetic iron particles have an average diameter of 35 nm to 1000 nm.

6: The antimicrobial particle of claim 1, wherein the magnetic iron particles have a concentration of between 0.1% w/w and 10% w/w.

7: The antimicrobial particle of claim 1, wherein the inorganic passivation layer comprises gallium oxide hydroxide (GaOOH) or gallium oxide (Ga₂O₃) or a combination thereof.

8: The antimicrobial particle of claim 1, wherein the inorganic passivation layer has a thickness of between 0.7 and 1.4 nm.

9: The antimicrobial particle of claim 1, wherein in response to a rotating magnetic field the particle is capable of becoming rod shaped, star shaped, spheroid shaped or a jagged sphere, is capable of fragmenting or a combination thereof.

10: A composition comprising one or more antimicrobial particles according to claim 1 and a carrier fluid.

11: The composition of claim 10, comprising at least one microparticle and at least one nanoparticle.

12: The composition of claim 10, wherein the carrier fluid is water.

13: A method of disrupting a biofilm, the method comprising:
contacting the biofilm with the composition according to claim 10; and
applying a magnetic field to the biofilm to magnetically activate the antibacterial particles and thereby disrupt the biofilm.

14: The method of claim 13, wherein the magnetic field is a rotating magnetic field.

15: The method of claim 14, wherein the rotational speed of the magnet is between 500 rpm and 2000 rpm.

16: The method of claim 13, further comprising contacting the biofilm simultaneously with an additional antimicrobial agent or contacting the disrupted biofilm with an additional antimicrobial agent.

17: The method of claim 13, wherein the biofilm is formed from bacteria and/or fungi.

18: A method of treating a biofilm related disease in a subject, the method comprising administering to the subject the composition according to claim 10;
and applying a magnetic field to the subject.

19: A process for forming a composition comprising antimicrobial particles, the process comprising:
(i) combining a liquid metal comprising gallium or an alloy thereof with magnetic iron particles to form a liquid metal ferrofluid, and
(ii) sonicating the liquid metal ferrofluid in an aqueous carrier fluid to form the antibacterial particles, wherein the antimicrobial particle comprises
a liquid metal core comprising
a liquid gallium or alloy thereof, and
a plurality of magnetic iron particles, and
an inorganic passivation layer encapsulating the liquid metal core.

20: The process of claim 19, wherein the liquid metal ferrofluid comprises 0.1% w/w to 10% w/w magnetic iron particles.
